(19) United States
(12) Patent Application Publication
(10) Pub. No.: US 2006/0259991 A1
(43) Pub. Date: Nov. 16, 2006

(54) Diagnostic and therapeutic use of scn2b protein for neurodegeneraative diseases

(75) Inventors: Heinz Von Der Kammer, Hamburg (DE); Johannes Pohlner, Hamburg (DE); Jozef Hanes, Breatislava IV (SK)

Correspondence Address: STERNE, KESSLER, GOLDSTEIN & FOX PLLC, 1100 NEW YORK AVENUE, N.W., WASHINGTON, DC, 20005, US

(73) Assignee: Evotech NeuroSciences GmbH, Hamburg (DE)

(21) Appl. No.: 10/543,314

(22) PCT Filed: Feb. 2, 2004
(86) PCT No.: PCT/EP04/00916

§ 371 Date: May 9, 2006

Related U.S. Application Data
(60) Provisional application No. 60/444,666, filed on Feb. 4, 2003.

(30) Foreign Application Priority Data
Feb. 4, 2003 (EP) 030002376.6

(51) Int. Cl. A01K 67/027; C12Q 1/68; G01N 33/567

(52) U.S. Cl. 800/12; 435/6; 435/7.2

(57) ABSTRACT

The present invention discloses the differential expression of the gene coding for SCN2B in specific brain regions of Alzheimer's disease patients. Based on this finding, the invention provides a method for diagnosing or prognosticating a neurodegenerative disease, in particular Alzheimer's disease, in a subject, or for determining whether a subject is at increased risk of developing such a disease. Furthermore, this invention provides therapeutic and prophylactic methods for treating or preventing Alzheimer's disease and related neurodegenerative disorders using the SCN2B gene and its corresponding gene products. A method of screening for modulating agents of neurodegenerative diseases is also disclosed.

The present invention relates to methods of diagnosing, prognosticating, and monitoring the progression of neurodegenerative diseases in a subject. Furthermore, methods of therapy control and screening for modulating agents of neurodegenerative diseases are provided. The invention also discloses pharmaceutical compositions, kits, and recombinant non-human animal models.

Neurodegenerative diseases, in particular Alzheimer's disease (AD), have a strongly debilitating impact on a patient's life. Furthermore, these diseases constitute an enormous health, social, and economic burden. AD is the most common neurodegenerative disease, accounting for about 70% of all dementia cases, and it is probably the most devastating age-related neurodegenerative condition affecting about 10% of the population over 65 years of age and up to 45% over age 85 (for a Presently, this amounts to an estimated 12 million cases in the US, Europe, and Japan. This situation will inevitably worsen with the demographic increase in the number of old people (“aging of the baby boomers”) in developed countries. The neuropathological hallmarks that occur in the brains of individuals with AD are senile plaques, composed of amyloid-β protein, and profound cytoskeletal changes coinciding with the appearance of abnormal filamentous structures and the formation of neurofibrillary tangles.

The amyloid-β (Aβ) peptide evolves, from the cleavage of the amyloid precursor protein (APP) by different kinds of proteases. The cleavage by the β/γ-secretase leads to the formation of Aβ peptides of different lengths, typically a short more soluble and slow aggregating peptide consisting of 40 amino acids and a longer 42 amino acid peptide, which rapidly aggregates outside the cells, forming the characteristic amyloid plaques (Selkoe, Physiological Rev 2001, 81-741-66; Greenfield et al., Frontiers Bioscience 2000, 5: D72-83). Two types of plaques, diffuse plaques and neuritic plaques, can be detected in the brain of AD patients, the latter ones being the classical, most prevalent type. They are primarily found in the cerebral cortex and hippocampus. The neuritic plaques have a diameter of 50 μm to 200 μm and are composed of insoluble fibrillar amyloids, fragments of dead neurons, of microglia and astrocytes, and other components such as neurotransmitters, apolipoprotein E, glycosaminoglycans, α1-antichymotrypsin and others. The generation of toxic Aβ deposits in the brain starts very early in the course of AD, and it is discussed to be a key player for the subsequent destructive processes leading to AD pathology. The other pathological hallmarks of AD are neurofibrillary tangles (NFTs) and abnormal neurites, described as neuropil threads (Braak and Braak, Acta Neuropathol. 1991, 82: 239-259). NFTs emerge inside neurons and consist of chemically altered tau, which forms paired helical filaments twisted around each other. Along the formation of NFTs, a loss of neurons can be observed. It is discussed that said neuron loss may be due to a damaged microtubule-associated transport system (Johnson and Jenkins, J Alzheimers Dis 1996, 1: 38-58; Johnson and Hartigan, J Alzheimers Dis 1999, 1: 329-351). The appearance of neurofibrillary tangles and their increasing number correlates well with the clinical severity of AD (Schmitt et al., Neurology 2000, 55: 370-376).

AD is a progressive disease that is associated with early deficits in memory formation and ultimately leads to the complete erosion of higher cognitive function. The cognitive disturbances include among other things memory impairment, aphasia, agnosia and the loss of executive functioning. A characteristic feature of the pathogenesis of AD is the selective vulnerability of particular brain regions and subpopulations of nerve cells to the degenerative process. Specifically, the temporal lobe region and the hippocampus are affected early and more severely during the progression of the disease. On the other hand, neurons within the frontal cortex, occipital cortex, and the cerebellum remain largely intact and are protected from neurodegeneration (Terry et al., Annals of Neurology 1981, 10: 184-92).

The age of onset of AD may vary within a range of 50 years, with early-onset AD occurring in people younger than 65 years of age, and late-onset of AD occurring in those older than 65 years. About 10% of all AD cases suffer from early-onset AD, with only 1-2% being familial, inherited cases.

Currently, there is no cure for AD, nor is there an effective treatment to halt the progression of AD or even to diagnose AD ante-mortem with high probability. Several risk factors have been identified that predispose an individual to develop AD, among them most prominently the epsilon 4 allele of the three different existing alleles (epsilon 2, 3, and 4) of the apolipoprotein E gene (ApoE) (Strittmatter et al., Proc Natl Acad Sci USA 1993, 90: 1977-81; Roses, Ann NY Acad Sci 1998, 855: 738-43). The polymorphic plasmaprotein ApoE plays a role in the intercellular cholesterol and phosphollpid transport by binding low-density lipoprotein receptors, and it seems to play a role in neurite growth and regeneration. Efforts to detect further susceptibility genes and disease-linked polymorphisms, lead to the assumption that specific regions and genes on human chromosomes 10 and 12 may be associated with late-onset AD (Myers et al., Science 2000, 290: 2304-5; Bertram et al., Science 2000, 290: 2303; Scott et al., Am J Hum Genet 2000, 66: 922-32).

Although there are rare examples of early-onset AD which have been attributed to genetic defects in the genes for amyloid precursor protein (APP) on chromosome 21, presenilin-1 on chromosome 14, and presenilin-2 on chromosome 1, the prevalent form of late-onset sporadic AD is of hitherto unknown etiologic origin. The late onset and complex pathogenesis of neurodegenerative disorders pose a formidable challenge to the development of therapeutic and diagnostic agents. It is crucial to expand the pool of potential drug targets and diagnostic markers. It is therefore an object of the present invention to provide insight into the pathogenesis of neurological-diseases and to provide methods, materials, agents, compositions, and non-human animal models which are suited inter alia for the diagnosis and development of a treatment of these diseases. This object has been solved by the features of the independent claims. The subclaims define preferred embodiments of the present invention.

The present invention is based on the differential expression of a gene coding for the voltage-gated sodium channel type II beta-subunit (beta-2 subunit, β2-subunit, β2 or SCN2B) in human Alzheimer's disease brain samples. Consequently, the SCN2B gene and its corresponding transcription and/or translation products may have a causative role in the regional selective neuronal degeneration typically observed in AD. Based on these disclosures, the present invention has utility for the diagnostic evaluation and prognosis as well as for the identification of a predisposition to a neurodegenerative disease, in particular AD. Furthermore, the present invention, provides methods for the diagnostic monitoring of patients undergoing treatment for such a disease.

Voltage-gated ion channels play an important role in the nervous system by generating conducted action potentials. Ion-conducting membrane channels for cations (sodium, calcium, potassium) and anions (chloride) are described (Lehmann-Horn et al., Physiological Reviews 1999, 79: 1317-1358). Transport of ions across the cell membrane leads to a fast transmission of electrical impulses throughout the cell network. Thereby, the channel switches between three functionally distinct states: a resting, an active, and an inactive one. Both, the resting and inactive states are nonconducting, and the channel is closed. As the membrane potential increases from less than −60 mV, the channel starts to open its pore (i.e. activation). Influx of ions (e.g. sodium) leads to a further increase of the membrane potential until an action potential is initiated. By closing the pore within 1 millisecond (i.e. fast inactivation) or within seconds to minutes (i.e. slow inactivation), the channel rapidly returns to an inactivated state. The ion conductance is highly's elective and efficient which enables fine tuning of processes such as memory, movement, and cognition (Lehmann-Horn et al., Physiological Reviews 1999, 79: 1317-1358). Molecular cloning of voltage-gated ion channels has uncovered a diversity of subtypes and enhanced the understanding about the underlying structure and function, particularly of sodium channels (Noda et al., Nature 1986, 322: 826-828; Schaller et al., Journal of Neuroscience 1995, 15: 3231-3242; Isom et al., Neuron 1994, 12: 1183-1194; Isom et al., Cell 1995, 83: 443-445).

Voltage-gated sodium channels are composed of a pore-forming α-subunit with approximately 260 kDa and of two smaller auxiliary β-subunits, β1 with ˜36 kDa and β2 with ˜33 kDa, which form a heteromeric complex. The α-subunit and both β-subunits are highly glycosylated (deglycosylated subunits with ˜220 kDa, ˜23 kDa, ˜21 kDa for α, β1 and β2, respectively). Three different genes encoding β-subunits have been identified to date. The β1-subunit is noncovalently associated with the α-subunit, whereas the β2-subunit is covalently attached to the a-subunit via a disulfide bridge. A third β-subunit isoform (β3), similar to the β1-subunit, has recently been discovered (Morgan et al., Proceedings National Academy of Science USA 2000, 97: 2308-2313). The α-subunit appears to be necessary and sufficient for sodium channel functionality. The β3-subunits modulate sodium channel function by accelerating the rate of inactivation, by increasing the amplitude Na⁺ peak current, by increasing the activation rate and by altering voltage dependency (Patton et al., Journal Biological Chemistry 1994, 269: 17649-17655). The β2-subunits appear not to be as effective as the β1-subunits, thus both may cooperate in modulating the function of the α-subunit.

The human β2-subunit is a glycoprotein with a signal peptide sequence of 29 amino acids, which is cleaved off during maturation of the protein. Additionally, the protein is made up of a short intracellular C-terminal segment, a single transmembrane region, and a large extracellular domain at the amino terminal, comprising conserved cysteine residues which are able to form a disulfide linkage with the. α-subunit. The β3-subunits exhibit an immunoglobulin-like motif, i.e. immunoglobulin-like C2-type domain, with structural similarities to cell adhesion molecules (CAM), for example contactin. Therefore, the β2-subunit might interact with extracellular matrix proteins such as tenascin-C and tenascin-R (Isom et al., Cell 1995, 83: 443-445). Cell adhesion molecules, such as the neuronal CAM (NCAM) and the myelin-associated glycoprotein (MAG), have immunoglobulin folds of the same type as the β2-subunit. The β2-subunits differ from the other β-subunits in that they feature a CAM motif and in the ability to locally expand the cell surface membrane. Thus, besides being modulators for sodium channels, they may function in cell-cell interaction; as regulators for sodium channel expression, and may regulate localization and immobilization of sodium channels in specific regions of the plasma membrane. Human β12 has similarity to myelin protein zero (p0), a transmembrane glycoprotein and a major structural component of peripheral myelin (Eubanks et al., Neuroreport 1997, 8: 2775-2779).

The genomic structure of the SCN2B gene was reported in 1998 (GenBank Accession No. AF049496; AF049497; Bolino et al., European Journal of Human Genetics 1998, 6: 629-634; and GenBank Accession No. AF049498; complete coding sequence and corresponding protein sequence). Mapping studies locate SCN2B to chromosome 11q23 (Eubanks et al., Neuroreport 1997, 8: 2775-2779). SCN2B covers approximately 12 kb of genomic DNA, harboring 4 exons and 3 introns which encode the mature SCN2B protein of 186 amino acids, and the SCN2B protein of 215 amino acids including the putative signal peptide of 29 amino acids (protein sequence and putative domains: GenBank Accession No. O60939). The human and rat SCN2B. proteins are of equal length and highly conserved (89% homology and 93% similarity).

Four brain sodium channels, i.e. SCN1A, SCN2A, SCN3A, and SCN8A, exhibit major expression in the central nervous system. The mRNAs of the β1 and β2-subunits (transcripts of ˜2 kb and ˜4 kb, respectively) have a broad but distinct regional expression pattern. In contrast to the β1-subunit encoding SCN1B gene, the SC-N2B gene Is expressed in the central nervous system only. Predominant expression levels were observed in the parahippocampal gyrus, the cortex, the granular layer, and Purkinje cells of the cerebellum. In rat brain, expression of β2 starts with embryonic day 9, in parallel with neurogenesis, and rapidly increases with axon extension and synaptogenesis (Isom et al.; Cell 1995, 83: 443-445).

For the neuronal sodium channel subunit genes SCN1A and SCN1B, disease-causing mutations leading to generalized epilepsy with febrile seizures plus (GEFS+) have been identified. Therefore, it is speculated that variations in the SCN2B gene may also result in common subtypes of idiopathic generalized epilepsy. Haug and coworkers identified a novel single nucleotide polymorphism (SNP) and a missense mutation (Asn209Pro) in the SCN2B gene, but excluded the SCN2B gene as a major susceptibility gene in epilepsy (Haug et al., Neuroreport 2000, 11: 2687-2689). Patent applications WO 02/19966, WO 02/087419, and WO 02/090532 implicate sodium channel β-subtypes with cardiovascular, inflammatory and muscular diseases, cancer and epilepsy. On the basis of protein homology and chromosomal localization, SCN2B has also been considered a suitable candidate gene for the demyelinating autosomal recessive motor and sensory neuropathy called Charcot-Marie-Tooth disease (CMT4B). This notion, however, was disputed by Bolino and coworkers (Bolino et al., European Journal of Human Genetics 1998, 6: 629-634). Nonetheless, patent application WO 01/79547 describes single nucleotide polymorphisms in the SCN2B gene which might be associated with demyelinating disorders. To date, there are no reports on a relationship between the β2-subunit of the voltage-gated ion channel type II and neurodegenerative disorders such as Alzheimer's disease,

Sodium channels are valuable targets for a variety of drugs as local anesthetics, anticonvulsants, antiarrythmics, for the treatment of neuropathic pain, epilepsy, and stroke. A number of toxins, drugs, and inorganic cations are used by the pharmaceutical industry as blockers of the α-subunit of sodium channels in central nervous system related disorders. Inhibitors of voltage-gated ion channels are already on the market, albeit they are of low potency, relatively non-specific, and their therapeutic potential is not fully exploited. Remarkably, no drugs are currently known that interact with the β-subunits of sodium channels. Thus, it is desirable to find specific drugs for a selective subunit of voltage-gated sodium channels such as the β-subunit.

The present invention discloses a dysregulation of voltage-gated sodium channel beta-2 subunit (β2-subunit, SCN2B) gene expression in brain samples from Alzheimer's disease patients. No such dysregulation is observed in samples derived from age-matched, healthy controls. To date, no experiments have been described that demonstrate a relationship between the dysregulation of β2-subunit gene expression and the pathology of Alzheimer's disease and related neurodegenerative disorders. Likewise, no mutations in the β2-subunit gene have been described to be associated with said diseases. Linking the β2-subunit gene to Alzheimer's disease, as disclosed in the instant invention, offers new ways, inter alia, for the diagnosis and treatment of said diseases.

The singular forms “a”, “an”, and “the” as used herein and in the claims include plural reference unless the context dictates otherwise. For example, “a cell” means as well a plurality of cells, and so forth. The term “and/or” as used in the present specification and in the claims implies that the phrases before and after this term are to be considered either as alternatives or in combination. For instance, the wording “determination of a level and/or an activity” means that either only a level, or only an activity, or both a level and an activity are determined. The term “level” as used herein is meant to comprise a gage of, or a measure of the amount of, or a concentration of a transcription product, for instance an mRNA, or a translation product, for instance a protein or polypeptide. The term “activity” as used herein shall be understood as a measure for the ability of a transcription product or a translation product to produce a biological effect or a measure for a level of biologically active molecules. The term “activity” also refers to enzymatic activity. The terms “level” and/or “activity” as used herein further refer to gene expression levels or gene activity. Gene expression can be defined as the utilization of the information contained in a gene by transcription and translation leading to the production of a gene product. “Dysregulation” shall mean an upregulation or downregulation of gene expression. A gene product comprises either RNA or protein and is the result of expression of a gene. The amount of a gene product can be used to measure how active a gene is. The term “gene” as used in the present specification and in the claims comprises both coding regions (exons) as well as non-coding regions (e.g. non-coding regulatory elements such as promoters or enhancers, introns, leader and trailer sequences). The term “ORF” is an acronym for “open reading frame” and refers to a nucleic acid sequence that does not possess a stop codon in at least one reading frame and therefore can potentially be translated into a sequence of amino acids. “Regulatory elements” shall comprise inducible and non-inducible promoters, enhancers, operators, and other elements that drive and regulate gene expression. The term “fragment” as used herein is meant to comprise e.g. an alternatively spliced, or truncated, or otherwise cleaved transcription product or translation product. The term “derivative” as used herein refers to a mutant, or an RNA-edited, or a chemically modified, or otherwise altered transcription product, or to a mutant, or chemically modified, or otherwise altered translation product. For instance, a “derivative” may be generated by processes such as altered phosphorylation, or glycosylation, or acetylation, or lipidation, or by altered signal peptide cleavage or other types of maturation cleavage. These processes may occur post-translationally. The term “modulator” as used in the present invention and in the claims refers to a molecule capable of changing or altering the level and/or the activity of a gene, or a transcription product of a gene, or a translation product of a gene. Preferably, a “modulator” is capable of changing or altering the biological activity of a transcription product or a translation product of a gene. Said modulation, for instance, may be an increase or a decrease in enzyme activity, a change in binding characteristics, or any other change or alteration in the biological, functional, or immunological properties of said translation product of a gene. The terms “agent”, “reagent”, or “compound” refer to any substance, chemical, composition or extract that have a positive or negative biological effect on a cell, tissue, body fluid, or within the context of any biological system, or any assay system examined. They can be agonists, antagonists, partial agonists or inverse agonists of a target. Such agents, reagents, or compounds may be nucleic acids, natural, or synthetic peptides or protein complexes, or fusion proteins. They may also be antibodies, organic or anorganic molecules or compositions, small molecules, drugs and any combinations of any of said agents above. They may be used for testing, for diagnostic or for therapeutic purposes. The terms “oligonucleotide primer” or “primer” refer to short nucleic acid sequences which can anneal to a given target polynucleotide by hybridization of the complementary base pairs and can be extended by a polymerase. They may be chosen to be specific to a particular sequence or they may be randomly selected, e.g. they will prime all possible sequences in a mix. The length of primers used herein may vary from 10 nucleotides to 80 nucleotides. “Probes” are short nucleic acid sequences of the nucleic acid sequences described and disclosed herein or sequences complementary therewith. They may comprise full length sequences, or fragments, derivatives, isoforms, or variants of a given sequence. The identification of hybridization complexes between a “probe” and an assayed sample allows the detection of the presence of other similar sequences within that sample. As used herein, “homolog or homology” is a term used in the art to describe the relatedness of a nucleotide or peptide sequence to another nucleotide or peptide sequence, which is determined by the degree of identity and/or similarity between said sequences compared. In the art, the terms “identity” and “similarity” mean the degree of polypeptide or polynucleotide sequence relatedness which are determined by matching a query sequence and other sequences of preferably the same type (nucleic acid or protein sequence) with each other. Preferred computer program methods to calculate and determine “identity” and “similarity” include, but are not-limited to GCG BLAST (Basic Local Alignment Search Tool) (Altschul et al., J. Mol. Biol. 1990, 215: 403-410; Altschul et al., Nucleic Acids Res. 1997, 25: 3389-3402; Devereux et al., Nucleic Acids Res. 1984, 12:,387), BLASTN 2.0 (Gish W., http://blast.wustl.edu, 1996-2002), FASTA (Pearson and Lipman, Proc. Natl. Acad. Sci. USA 1988, 85: 2444-2448), and GCG GelMerge which determines and aligns a pair of contigs with the longest overlap (Wilbur and Lipman, SIAM J. Appl. Math. 1984, 44: 557-567; Needleman and Wunsch, J. Mol. Biol. 1970, 48: 443-453). The term “variant” as used herein refers to any polypeptide or protein, in reference to polypeptides and proteins disclosed in the present invention, in which one or more amino acids are added and/or substituted and/or deleted and/or inserted at the N-terminus, and/or the C-terminus, and/or within the native amino acid sequences of the native polypeptides or proteins of the present invention, but retains its essential properties. Furthermore, the term “variant” shall include any shorter or longer version of a polypeptide or protein. “Variants” shall also comprise a sequence that has at least about 80% sequence identity, more preferably at least about 90% sequence identity, and most preferably at least about 95% sequence identity with the amino acid sequences of a voltage-gated sodium channel beta-subunit protein, in particular SCN2B, SEQ ID NO. 1. “Variants” of a protein molecule shown in SEQ ID NO. 1 include, for example, proteins with conservative amino acid substitutions in highly conservative regions. “Proteins and polypeptides” of the present invention include variants, fragments and chemical derivatives of the protein comprising the amino acid sequences of SCN2B, SEQ ID NO. 1. They can include proteins and polypeptides which can be isolated from nature or be produced by recombinant and/or synthetic means. Native proteins or polypeptides refer to naturally-occurring truncated or secreted forms, naturally occurring variant forms (e.g. splice-variants) and naturally occurring allelic variants. The term “isolated” as used herein is considered to refer to molecules or substances which have been changed and/or which have been removed from their natural environment, i.e. isolated from a cell or from a living organism in which they normally occur, and that are separated or essentially purified from the coexisting components with which they are found to be associated in nature. This notion further means that the sequences encoding such molecules can be linked by the hand of man to polynucleotides, to which they are not linked in their natural state, and that such molecules can be produced by recombinant and/or synthetic means. Even if for said purposes those sequences may be introduced into living or non-living organisms by methods known to those skilled in the art, and even if those sequences are still present in said organisms, they are still considered to be isolated. In the present invention, the terms “risk”, “susceptibility”, and “predisposition” are tantamount and are used with respect to the probability of developing a neurodegenerative disease, preferably Alzheimer's disease.

The term ‘AD’ shall mean Alzheimer's disease. “AD-type neuropathology” as used herein refers to neuropathological, neurophysiological, histopathological and clinical hallmarks as described in the instant invention and as commonly known from state-of-the-art literature (see: lqbal, Swaab, Winblad and Wisniewski, Alzheimer's Disease and Related Disorders (Etiology, Pathogenesis and Therapeutics), Wiley & Sons, New York, Weinheim, Toronto, 1999; Scinto and Daffner, Early Diagnosis of Alzheimer's Disease, Humana Press, Totowa, N.J., 2000; Mayeux and Christen, Epidemiology of Alzheimer's Disease: From Gene to Prevention, Springer Press, Berlin, Heidelberg, N.Y., 1999; Younkin, Tanzi and Christen, Presenilins and Alzheimer's Disease, Springer Press, Berlin, Heidelberg, N.Y., 1998). Neurodegenerative diseases or disorders according, to the present invention comprise Alzheimer's disease, Parkinson's disease, Huntington's disease, amyotrophic lateral sclerosis, Pick's disease, fronto-temporal dementia, progressive nuclear palsy, corticobasal degeneration, cerebro-vascular dementia, multiple system atrophy, argyrophilic grain dementia and other tauopathies, and mild-cognitive impairment. Further conditions involving neurodegenerative processes are, for instance, ischemic stroke, age-related macular degeneration, narcolepsy, motor neuron diseases, prion diseases, traumatic nerve injury and repair, and multiple sclerosis.

In one aspect, the invention features a method of diagnosing or prognosticating a neurodegenerative disease in a subject, or determining whether a subject is at increased risk of developing said disease. The method comprises: determining a level, or an activity, or both said level and said activity of (i) a transcription product of the gene coding for SCN2B, and/or of (ii) a translation product of the gene coding for SCN2B, and/or of (iii) a fragment, or derivative, or variant of said transcription or translation product in a sample from said subject and comparing said level, and/or said activity to a reference value representing a known disease or health status, thereby diagnosing or prognosticating said neurodegenerative disease in said subject, or determining whether said subject is at increased risk of developing said neurodegenerative disease.

The invention also relates to the construction and the use of primers and probes which are unique to the nucleic acid sequences, or fragments, or variants thereof, as disclosed in the present invention. The oligonucleotide primers and/or probes can be labeled specifically with fluorescent, bioluminescent, magnetic, or radioactive substances. The invention further relates to the detection and the production of said nucleic acid sequences, or fragments and variants thereof, using said specific oligonucleotide primers in appropriate combinations. PCR-analysis, a method well known to those skilled in the art, can be performed with said primer combinations to amplify said gene specific nucleic acid sequences from a sample containing nucleic acids. Such sample may be derived either from healthy or diseased subjects. Whether an amplification results in a specific nucleic acid product or not, and whether a fragment of different length can be obtained or not, may be indicative for a neurodegenerative disease, in particular Alzheimer's disease. Thus, the invention provides nucleic acid sequences, oligonucleotide primers, and probes of at least 10 bases in length up to the entire coding and gene sequences, useful for the detection of gene mutations and single nucleotide polymorphisms in a given sample comprising nucleic acid sequences to be examined, which may be associated with neurodegenerative diseases, in particular Alzheimer's disease. This feature has utility for developing rapid DNA-based diagnostic tests, preferably also in the format of a kit.

In a further aspect, the invention features a method of monitoring the progression of a neurodegenerative disease in a subject. A level, or an activity, or both said level and said activity, of (i) a transcription product of the gene coding for SCN2B, and/or of (ii) a translation product of the gene coding for SCN2B, and/or of (iii) a fragment, or derivative, or variant of said transcription or translation product in a sample from said subject is determined. Said level and/or said activity is compared to a reference value representing a known disease or health status. Thereby, the progression of said neurodegenerative disease in said subject is monitored.

In still a further aspect, the invention features a method of evaluating a treatment for a neurodegenerative disease, comprising determining a level, or an activity, or both said level and said activity of (i) a transcription product of the gene coding for SCN2B, and/or of (ii) a translation product of the gene coding for SCN2B, and/or of (iii) a fragment, or derivative, or variant of said transcription or translation product in a sample obtained from a subject being treated for said disease. Said level, or said activity, or both said level and said activity are compared to a reference value representing a known disease or health status, thereby evaluating the treatment for said neurodegenerative disease.

In a preferred embodiment of the herein claimed methods, kits, recombinant non-human animals, molecules, assays, and uses of the instant invention, said gene coding for a human voltage-gated sodium channel beta subunit protein is the gene coding for a human voltage-gated sodium channel type II beta subunit (SCN2B), also termed beta-2 subunit protein or β2-protein represented by the gene coding for the protein of Genbank accession number O60939 (protein ID), SEQ ID NO. 1, which is deduced from the mRNA corresponding to the cDNA sequences of Genbank accession numbers AF049498, AF049496, AF049497, AF107028, AX376000 and ESTs from the Genbank database, SEQ ID NO. 2. In the instant invention SCN2B also refers to the nucleic acid sequence of SEQ ID NO. 2, coding for the protein of SEQ ID NO. 1 (Genbank accession number O60939). In the instant invention said sequences are “isolated” as the term is employed herein. Further, in the instant invention, the gene coding for said SCN2B protein is also generally referred to as the SCN2B gene, or simply SCN2B, and the protein of SCN2B is also generally referred to as the SCN2B protein, or simply SCN2B.

In a further preferred embodiment of the herein claimed methods, kits, recombinant non-human animals, molecules, assays, and uses of the instant invention, said neurodegenerative disease or disorder is Alzheimer's disease, and said subjects suffer from Alzheimer's disease.

The present invention discloses the detection and differential expression and regulation of the gene coding for SCN2B in specific brain regions of AD patients.

Consequently, the SCN2B gene and its corresponding transcription and/or translation products may have a causative role in the regional selective neuronal degeneration typically observed in AD. Alternatively, SCN2B may confer a neuroprotective function to the remaining surviving nerve cells. Based on these disclosures, the present invention has utility for the diagnostic evaluation and prognosis as well as for the identification of a predisposition to a neurodegenerative disease, in particular AD. Furthermore, the present invention provides methods for the diagnostic monitoring of patients undergoing treatment for such a disease.

It is preferred that the sample to be analyzed and determined is selected from the group comprising brain tissue, or other tissues, or body cells. The sample can also comprise cerebrospinal fluid or other body fluids including saliva, urine, blood, serum plasma, or mucus. Preferably, the methods of diagnosis, prognosis, monitoring the progression or evaluating a treatment for a neurodegenerative disease, according to the instant invention, can be practiced ex corpore, and such methods preferably relate to samples, for instance, body fluids or cells, removed, collected, or isolated from a subject or patient.

In further preferred embodiments, said reference value is that of a level, or an activity, or both said level and said activity of (i) a transcription product of the gene coding for SCN2B, and/or of (ii) a translation product of the gene coding for SCN2B, and/or of (iii) a fragment, or derivatives or variant of said transcription or translation product in a sample from a subject not suffering from said neurodegenerative disease.

In preferred embodiments, an alteration in the level and/or activity of a transcription product of the gene coding for SCN2B and/or of a translation product of the gene coding for SCN2B and/or of a fragment, or derivative, or variant thereof in a sample cell, or tissue, or body fluid from said subject relative to a reference value representing a known health status indicates a diagnosis, or prognosis, or increased risk of becoming diseased with a neurodegenerative disease, particularly AD.

In preferred embodiments, measurement of the level of transcription products of the SCN2B gene is performed in a sample from a subject using a quantitative PCR-analysis with primer combinations to amplify said gene specific sequences from cDNA obtained by reverse transcription of RNA extracted from a sample of a subject. A Northern blot with probes specific for said gene can also be applied. It might further be preferred to measure transcription products by means of chip-based microarray technologies. These techniques are known to those of ordinary skill in the art (see Sambrook and Russell, Molecular Cloning: A Laboratory Manual, Cold Spring Harbor Laboratory Press, Cold Spring Harbor, N.Y., 2001; Schena M., Microarray Biochip Technology, Eaton Publishing, Natick, Mass., 2000). An example of an immunoassay is the detection and measurement of enzyme activity as disclosed and described in the patent application WO 02/14543.

Furthermore, a level and/or an activity of a translation product of the gene coding for SCN2B and/or of a fragment, or derivative, or variant of said translation product, and/or the level of activity of said translation product, and/or of a fragment, or derivative, or variant thereof, can be detected using an immunoassay, an activity assay, and/or a binding assay. These assays can measure the amount of binding between said protein molecule and an anti-protein antibody by the use of enzymatic, chromodynamic, radioactive, magnetic, or luminescent labels which are attached to either the anti-protein antibody or a secondary antibody which binds the anti-protein antibody. In addition, other high affinity ligands may be used. Immunoassays which can be used include e.g. ELISAs, Western blots, -and other techniques known to those of ordinary skill in the art (see Harlow and Lane, Using Antibodies: A Laboratory Manual, Cold Spring Harbor Laboratory Press, Cold Spring Harbor, N.Y., 1999 and Edwards R, Immunodiagnostics: A Practical Approach, Oxford University-Press, Oxford; England, 1999). All these detection techniques may also be employed in the format of microarrays, protein-arrays, antibody microarrays, tissue microarrays, electronic biochip or protein-chip based technologies, (see Schena M., Microarray Biochip Technology, Eaton Publishing, Natick, Mass.; 2000).

In a preferred embodiment, the level, or the activity, or both said level and said activity of (i) a transcription product of the gene coding for SCN2B, and/or of (ii) a translation product of the gene coding for SCN2B, and/or of (iii) a fragment, or derivative, or variant of said transcription or translation product in a series of samples taken from said subject over a period of time is compared, in order to monitor the progression of said disease. In further preferred embodiments, said subject receives a treatment prior to one or more of said sample gatherings. In yet another preferred embodiment, said level and/or activity is determined before and after said treatment of said subject.

In another aspect, the invention features a kit for diagnosing or prognosticating neurodegenerative diseases, in particular AD, in a subject, or determining the propensity or predisposition of a subject to develop a neurodegenerative disease, in particular AD, said kit comprising:

(a) at least one reagent which-is selected from the group consisting of (i) reagents that selectively detect a transcription product of the gene coding for SCN2B, and (ii) reagents that selectively detect a translation product of the gene coding for SCN2B; and

(b) an instruction for diagnosing, or prognosticating a neurodegenerative disease, in particular AD, or determining the propensity or predisposition of a subject to develop such a disease by

detecting a level, or an activity, or both said level and said activity, of said transcription product and/or said translation product of the gene coding for SCN2B, in a sample from said subject; and

diagnosing or prognosticating a neurodegenerative disease, in particular AD, or determining the propensity or predisposition of said subject to develop such a disease, wherein a varied level, or activity, or both said level and said activity, of said transcription product and/or said translation product compared to a reference value representing a known health status; or a level, or activity, or both said level and said activity, of said transcription product and/or said translation product similar or equal to a reference value representing a known disease status, indicates a diagnosis or prognosis of a neurodegenerative disease, in particular AD, or an increased propensity or predisposition of developing such a disease. The kit, according to the present invention, may be particularly useful for the identification of individuals that are at risk of developing a neurodegenerative disease, in particular AD. Consequently, the kit, according to the invention, may serve as a means for targeting identified individuals for early preventive measures or therapeutic intervention prior to disease onset, before irreversible damage in the course of the disease has been inflicted. Furthermore, in preferred embodiments, the kit featured in the invention is useful for monitoring a progression of a neurodegenerative disease, in particular AD, in a subject, as well as monitoring success or failure of therapeutic treatment for such a disease of said subject.

In another aspect, the invention features a method of treating or preventing a neurodegenerative disease, in particular AD, in a subject comprising the administration to said subject in a therapeutically or prophylactically effective amount of an agent or agents which directly or indirectly affect a level, or an activity, or both said level and said activity, of (i) the gene coding for SCN2B, and/or (ii) a transcription product of the gene coding for SCN2B, and/or (iii) a translation product of the gene coding for SCN2B, and/or (iv) a fragment, or derivative, or variant of (i) to (iii). Said agent may comprise a small molecule, or it may also comprise a peptide, an oligopeptide, or a polypeptide. Said peptide, oligopeptide, or polypeptide may comprise an amino acid sequence of a translation product of the gene coding for SCN2B protein, or a fragment, or derivative, or a variant thereof. An agent for treating or preventing a neurodegenerative disease, in particular AD, according to the instant invention, may also consist of a nucleotide, an oligonucleotide, or a polynucleotide. Said oligonucleotide or polynucleotide may comprise a nucleotide sequence of the gene coding for SCN2B protein, either in sense orientation or in antisense orientation.

In preferred embodiments, the method comprises the application of per se known methods of gene therapy and/or antisense nucleic acid technology to administer said agent or agents. In general, gene therapy includes several approaches: molecular replacement of a mutated gene, addition of a new gene resulting in the synthesis of a therapeutic protein, and modulation of endogenous cellular gene expression by recombinant expression methods or by drugs. Gene-transfer techniques are described in detail (see e.g. Behr, Acc Chem Res 1993, 26: 274-278 and Mulligan, Science 1993, 260: 926-931) and include direct gene-transfer techniques such as mechanical microinjection of DNA into a cell as well as indirect techniques employing biological vectors (like recombinant viruses, especially retroviruses) or model liposomes, or techniques based on transfection with DNA coprecipitation with polycations, cell membrane pertubation by chemical (solvents, detergents, polymers, enzymes) or physical means (mechanic, osmotic, thermic, electric shocks). The postnatal gene transfer into the central nervous system has been described in detail (see e.g. Wolff, Curr Opin Neurobiol 1993, 3: 743-748).

In particular, the invention features a method of treating or preventing a neurodegenerative disease by means of antisense nucleic acid therapy, i.e. the down-regulation of an inappropriately expressed or defective gene by the introduction of antisense nucleic acids or derivatives thereof into certain critical cells (see e.g. Gillespie, DN&P 1992, 5: 389-395; Agrawal and Akhtar, Trends Biotechnol 1995, 13: 197-199; Crooke, Biotechnology 1992, 10: 882-6). Apart from hybridization strategies, the application of ribozymes, i.e. RNA molecules that act as enzymes, destroying RNA that carries the message of disease has also been described (see e.g. Barinaga, Science 1993, 262: 1512-1514). In preferred embodiments, the subject to be treated is a human, and therapeutic antisense nucleic acids or derivatives thereof are directed against transcripts of the gene coding for SCN2B. It is preferred that cells of the central nervous system, preferably the brain, of a subject are treated in such a way. Cell penetration can be performed by known strategies such as coupling of antisense nucleic acids and derivatives thereof to carrier particles, or the above described techniques. Strategies for administering targeted therapeutic oligodeoxynucleotides are known to those of skill in the art (see e.g. Wickstrom, Trends Biotechnol 1992, 10: 281-287). In some cases, delivery can be performed by mere topical application. Further approaches are directed to intracellular expression of antisense RNA. In this strategy, cells are transformed ex vivo with a recombinant gene that directs the synthesis of an RNA that is complementary to a region of target nucleic acid. Therapeutical use of intracellularly expressed antisense RNA is procedurally similar to gene therapy. A recently developed method of regulating the intracellular expression of genes by the use of double-stranded RNA, known variously as RNA interference (RNAi), can be another effective approach for nucleic acid therapy (Hannon, Nature 2002, 418: 244-251).

In a further preferred embodiment, a method to investigate the effects of compounds and/or agents on SCN2B coexpressed with the voltage-gated sodium channel SCN2A or other voltage-gated sodium channel proteins in appropriate cells, for example CHO cells or HEK293 cells, or other neuronal, cell lines, is provided. Thereby, the electrophysiological effect of compounds and/or agents on the sodium current mediated by SCN2B coexpressed with SCN2A or with other voltage-gated sodium channel proteins is examined. To conduct said examination the cDNA coding for human gene product SCN2B is cloned into an appropriate expression-vector. The cDNA coding for SCN2A (Genbank accession numbers AF327224-AF327246), or for other voltage-gated sodium channels, is cloned into another appropriate expression-vector. Appropriate cell lines, as mentioned above, are transfected with said plasmids, preferably using a reagent like DMRIE-C (liposome formulation of the cationic lipid 1,2-dimyristyloxypropyl-3-dimethyl-hydroxy ethyl ammonium bromide-chloesterol). Patch-clamp experiments can be performed in the voltage-clamp mode (Hamill et al., Pflügers Arch. 1981, 391: 85-100), and whole-cell currents will be recorded, and the obtained signals will be amplified, digitized, stored and analyzed using an appropriate software, for example Pulse/Pulsefit (HEKA, Lambrecht, Germany). If current “run-down” or “run-up” (Varnum et al., Pro. Natl. Acad. Sci. USA 1993, 90: 11528-11532) remains to be too strong for compound and/or agent effect evaluation, investigations on the mediated currents of said voltage-gated sodium channels can be performed with the perforated patch-clamp method to prevent unspecific current amplitude changes (Dart et al., J. Physiol. 1995, 483: 29-39; binesh & Hablitz, Brain Res. 1990, 535: 318-322). An example of a stimulation protocol for the investigation of the effects and reversibility of test compounds on SCN2B coexpressed with SCN2A, or other sodium channel proteins, is given below. Cells coexpressing SCN2B with SCN2A, or other sodium channels, will be clamped at a holding potential of e.g. −80 mV. The pulse cycling rate may be 10 s. For the compound and/or agent testing, stably transfected cells can be hyperpolarized from a holding potential of e.g. −80 mV for e.g. 100 ms to e.g. −100 mV or −120 mV, followed by, for instance, a 1s depolarization to a test potential between −60 mV and +60 mV. The peak current amplitude of the test pulse will be used for the analysis. The method is also suitable to identify and test compounds and/or agents which are capable for opening, closing, activating, inactivating, or modifying the biophysical properties of SCN2B coexpressed with SCN2A or other voltage-gated sodium channels. Modulators of voltage-gated sodium channels, thus identified and tested, can potentially influence learning and memory functions and can be used for therapeutic approaches, for example for neurodegenerative diseases, in particular for Alzheimer's disease.

In further preferred embodiments, the method comprises grafting donor cells into the central nervous system, preferably the brain, of said subject, or donor cells preferably treated so as to minimize or reduce graft rejection, wherein said donor cells are genetically modified by insertion of at least one transgene encoding said agent or agents. Said transgene might be carried by a viral vector, in particular a retroviral vector. The transgene can be inserted into the donor cells by a nonviral physical transfection of DNA encoding a transgene, in particular by microinjection. Insertion of the transgene can also be performed by electroporation, chemically mediated transfection, in particular calcium phosphate transfection, or liposomal mediated transfection (see Mc Celland and Pardee, Expression Genetics: Accelerated and High—Throughput Methods, Eaton Publishing, Natick, Mass., 1999).

In preferred embodiments, said agent for treating and preventing a neurodegenerative disease, in particular AD, is a therapeutic protein which can be administered to said subject, preferably a human, by a process comprising introducing subject cells into said subject, said subject cells having been treated in vitro to insert a DNA segment encoding said therapeutic protein, said subject cells expressing in vivo in said subject a therapeutically effective amount of said therapeutic protein. Said DNA segment can be inserted into said cells in vitro by a viral vector, in particular a retroviral vector.

Methods of treatment, according to the present invention, comprise the application of therapeutic cloning, transplantation, and stem cell therapy using embryonic stem cells or embryonic germ cells and neuronal adult stem cells, combined with any of the previously described cell—and gene therapeutic methods. Stem cells may be totipotent or pluripotent. They may also be organ-specific. Strategies for repairing diseased and/or damaged brain cells or tissue comprise (i) taking donor cells from an adult tissue. Nuclei of those cells are transplanted into unfertilized egg cells from which the genetic material has been removed. Embryonic stem cells are isolated from the blastocyst stage of the cells which underwent somatic cell nuclear transfer. Use of differentiation factors then leads to a directed development of the stem cells to specialized cell types, preferably neuronal cells (Lanza et al., Nature Medicine 1999, 9: 975-977), or (ii) purifying adult stem cells, isolated from the central nervous system, or from bone marrow (mesenchymal stem cells), for in vitro expansion and subsequent grafting and transplantation, or (iii) directly inducing endogenous neural stem cells to proliferate, migrate, and differentiate into functional neurons (Peterson DA, Curr. Opin. Pharmacol. 2002, 2: 34-42). Adult neural stem cells are of great potential for repairing damaged or diseased brain tissues, as the germinal centers of the adult brain are free of neuronal damage or dysfunction (Colman A, Drug Discovery World 2001, 7: 66-71).

In preferred embodiments, the subject for treatment or prevention, according to the present invention, can be a human, an experimental animal, e.g. a mouse or a rat, a domestic animal, or a non-human primate. The experimental animal can be a non-human animal model for a neurodegenerative disorder, e.g. a transgenic mouse and/or a knock-out mouse with an AD-type neuropathology.

In a further aspect, the invention features a modulator of an activity, or a level, or both said activity and said level of at least one substance which is selected from the group consisting of (i) the gene coding for SCN2B, and/or (ii) a transcription product of the gene coding for. SCN2B and/or (iii) a translation product of the gene coding for SCN2B, and/or (iv) a fragment, or derivative, or variant of (i) to (iii).

In an additional aspect, the invention features a pharmaceutical composition comprising said modulator and preferably a pharmaceutical carrier. Said carrier refers to a diluent, adjuvant, excipient, or vehicle with which the modulator is administered.

In a further aspect, the invention features a modulator of an activity, or a level, or both said activity and said level of at least one substance which is selected from the group consisting of (i) the gene coding for SCN2B, and/or (ii) a transcription product of the gene coding for SCN2B, and/or (iii) a translation product of the gene coding for SCN2B, and/or (iv) a fragment, or derivative, or variant of (i) to (iii) for use in a pharmaceutical composition.

In another aspect, the invention provides for the use of a modulator of an activity, or a level, or both said activity and said level of at least one substance which is selected from the group consisting of (i) the gene coding for SCN2B, and/or (ii) a transcription product of the gene coding for SCN2B and/or (iii) a translation product of the gene coding for SCN2B, and/or (iv) a fragment, or derivative, or variant of (i) to (iii) for a preparation of a medicament for treating or preventing a neurodegenerative disease, in particular AD.

In one aspect, the present invention also provides a kit comprising one or more containers filled with a therapeutically or prophylactically effective amount of said pharmaceutical composition.

In a further aspect, the invention features a recombinant, non-human animal comprising a non-native gene sequence coding for SCN2B, or a fragment, or a derivative, or variant thereof. The generation of said recombinant, non-human animal comprises (i) providing a gene targeting construct containing said gene sequence and a selectable marker sequence, and (ii) introducing said targeting construct into a stem cell of a non-human animal, and (iii) introducing said non-human animal stem cell into a non-human embryo, and (iv) transplanting said embryo into a pseudopregnant non-human animal, and (v) allowing said embryo to develop to term, and (vi) identifying a genetically altered non-human animal whose genome comprises a modification of said gene sequence in both alleles, and (vii) breeding the genetically altered non-human animal of step (vi) to obtain a genetically altered non-human animal whose genome comprises a modification of said endogenous gene, wherein said gene is mis-expressed, or under-expressed, or over-expressed, and wherein said disruption or alteration results in said non-human animal exhibiting a predisposition to developing symptoms of neuropathology similar to a neurodegenerative disease, in particular AD. Strategies and techniques for the generation and construction of such an animal are known to those of ordinary skill in the art (see e.g. Capecchi, Science 1989, 244: 1288-1292; Hogan et al., Manipulating the Mouse Embryo: A Laboratory Manual, Cold Spring Harbor Laboratory Press, Cold Spring Harbor, N.Y., 1994 and Jackson and Abbott, Mouse Genetics and Transgenics: A Practical Approach, Oxford University Press, Oxford, England, 1999). It is preferred to make use of such a recombinant non-human animal as an animal model for investigating neurodegenerative diseases, in particular AD. Such an animal may be useful for screening, testing and validating compounds, agents and modulators in the development of diagnostics and therapeutics to treat neurodegenerative diseases, in particular Alzheimer's disease. In preferred embodiments, said recombinant, non-human animal comprises a non-native gene sequence coding for SCN2B protein, or a fragment, or derivative, or variant thereof.

In another aspect, the invention features an assay for screening for a modulator of neurodegenerative diseases, in particular AD, or related diseases and disorders of one or more substances selected from the group consisting of (i) the gene coding for SCN2B, and/or (ii) a transcription product of the gene coding for SCN2B, and/or (iii). a translation product of the gene coding for SCN2B, and/or (iv) a fragment, or derivative, or variant of (i) to (iii). This screening method comprises (a) contacting a cell with a test compound, and (b) measuring the activity, or the level, or both the activity and the level of one or more substances recited in (i) to (iv), and (c) measuring the activity, or the level, or both the activity and the level of said substances in a control cell not contacted with said test compound, and (d) comparing the levels of the substance in the cells of step (b) and (c), wherein an alteration in the activity and/or level of said substances in the contacted cells indicates that the test compound is a modulator of said diseases and disorders.

In one further aspect, the invention features a screening assay for a modulator of neurodegenerative diseases, in particular AD, or related diseases and disorders of one or more substances selected from the group consisting of (i) the gene coding for SCN2B, and/or (ii) a transcription product of the gene coding for SCN2B, and/or (iii) a translation product of the gene coding for SCN2B, and/or (iv) a fragment, or derivative, or variant of (i) to (iii), comprising (a) administering a test compound to a non-human test animal which is predisposed to developing or has already developed symptoms of a neurodegenerative disease or related diseases or disorders, and (b) measuring the activity and/or level of one or more substances recited in (i) to (iv), and (c) measuring the activity and/or level of said substances in a matched non-human control animal which is equally predisposed to developing or has already developed said symptoms and to which non-human animal no such test compound has been administered, and (d) comparing the activity and/or level of the substance in the animals of step (b) and (c), wherein an alteration in the activity and/or level of substances in the non-human test animal indicates that the test compound is a modulator of said diseases and disorders.

In a preferred embodiment, said non-human test animal and/or said non-human control animal is a recombinant, non-human animal which expresses the gene coding for SCN2B, or a fragment thereof, or a derivative, or a variant thereof, under the control of a transcriptional regulatory element which is not the native SCN2B gene transcriptional control regulatory element.

In another embodiment, the present invention provides a method for producing a medicament comprising the steps of (i) identifying a modulator of neurodegenerative diseases by a method of the aforementioned screening assays and (ii) admixing the modulator with a pharmaceutical carrier. However, said modulator may also be identifiable by other types of screening assays.

In another aspect, the present invention provides for an assay for testing a compound, preferably for screening a plurality of compounds, for inhibition of binding between a ligand and a translation product of the gene coding for SCN2B, or a fragment, or derivative, or variant thereof. Said screening assay comprises the steps of (i) adding a liquid suspension of said SCN2B translation product, or a fragment or derivative thereof, to a plurality of containers, and (ii) adding a compound or a plurality of compounds to be screened for said inhibition to said plurality of containers, and (iii) adding a detectable, preferably a fluorescently labelled ligand to said containers, and (iv) incubating said SCN2B translation product, or said, fragment, or derivative, or variant thereof, and said compound or plurality of compounds, and said detectable, preferably fluorescently labelled ligand, and (v) measuring the amounts of preferably the fluorescence associated with said SCN2B translation product, or with said fragment, or derivative, or variant thereof, and (vi) determining the degree of inhibition by one or more of said compounds of binding of said ligand to said SCN2B translation product, or said fragment, or derivative, or variant thereof. It might be preferred to reconstitute said SCN2B translation product, or fragment, or derivative, or variant thereof into artificial liposomes to generate the corresponding proteoliposomes to determine the inhibition of binding between a ligand and said SCN2B translation product. Methods of reconstitution of SCN2B translation products from detergent into liposomes have been detailed (Schwarz et al., Biochemistry 1999, 38: 9456-9464; Krivosheev and Usanov, Biochemistry-Moscow 1997, 62: 1064-1073). Instead of utilizing a fluorescently labelled ligand, it might in some aspects be preferred to use any other detectable label known to the person skilled in the art, e.g. radioactive labels, and detect it accordingly. Said method may be useful for the identification of novel compounds as well as for evaluating compounds which have been improved or otherwise optimized in their ability to inhibit the binding of a ligand to a gene product of the gene coding for SCN2B, or a fragment, or derivative, or variant thereof. One example of a fluorescent binding assay, in this case based on the use of carrier particles, is disclosed and described in patent application WO 00/52451. A further example is the competitive assay method as described in patent WO 02/01226. Preferred signal detection methods for the screening assays of the instant invention are described in the following patent applications: WO 96/13744, WO 98/16814, WO 98/23942,WO 99/17086, WO 99/34195, WO 00/66985, WO 01/59436, WO 01/59416.

In one further embodiment, the present invention provides a method for producing a medicament comprising the steps of (i) identifying a compound as an inhibitor of binding between a ligand and a gene product of the gene coding for SCN2B by the aforementioned inhibitory binding assay and (ii) admixing the compound with a pharmaceutical carrier. However, said compound may also be identifiable by other types of screening assays.

In another aspect, the invention features an assay for testing a compound, preferably for screening a plurality of compounds to determine the degree of binding of said compounds to a translation product of the gene coding for SCN2B, or to a fragment, or derivative, or variant thereof. Said screening assay comprises (i). adding a liquid suspension of said SCN2B translation product, or a fragment, or derivative, or variant thereof, to a plurality of containers, and (ii,) adding a detectable, preferably a fluorescently labelled compound or a plurality of detectable, preferably fluorescently labelled compounds to be screened for said binding to said plurality of containers, and (iii) incubating said SCN2B translation product, or said fragment, or derivative, or variant thereof, and said detectable, preferably fluorescently labelled compound or detectable, preferably fluorescently labelled compounds, and (iv) measuring the amounts of preferably the fluorescence associated with said SCN2B translation product, or with said fragment, or derivative, or variant thereof, and (v) determining the degree of binding by one or more of said compounds to said SCN2B translation product, or said fragment, or derivative, or variant thereof. In this type of assay it might be preferred to use a fluorescent label. However, any other type of detectable label might also be employed. Also in this type of assay it might be preferred to reconstitute a SCN2B translation product or fragment, or derivative, or variant thereof into artificial liposomes as described in the present invention, Said assay methods may be useful for the identification of hovel compounds as well as for evaluating compounds which have been improved or otherwise optimized in their ability to bind to a SCN2B translation product, or fragment, or derivative, or variant thereof.

In one further embodiment, the present invention provides a method for producing a medicament comprising: the steps of (i) identifying a compound as a binder to the gene product of the SCN2B gene by the aforementioned binding assays and (ii) admixing the compound with a pharmaceutical carrier. However, said compound may also be identifiable by other types of screening assays.

In another embodiment, the present invention provides for a medicament obtainable by any of the methods according to the herein claimed screening assays. In one further embodiment, the instant invention provides for a medicament obtained by any of the methods according to the herein claimed screening assays.

The present invention features a protein molecule shown in SEQ ID NO. 1, said protein molecule being a translation product of the gene coding for SCN2B, or a fragment, or derivative, or variant thereof, for use as a diagnostic target for detecting a neurodegenerative disease, preferably Alzheimer's disease.

The present invention further features a protein molecule shown in SEQ ID NO.1, said protein molecule being a translation product of the gene coding for SCN2B, or a fragment, or derivative, or variant thereof, for use as a screening target for reagents or compounds preventing, or treating, or ameliorating a neurodegenerative disease, preferably Alzheimer's disease.

The present invention features an antibody which is specifically immunoreactive with an immunogen, wherein said immunogen is a translation product of the gene coding for SCN2B, SEQ ID NO.1, or a fragment, or variant, or derivative thereof. The immunogen may comprise immunogenic or antigenic epitopes or portions of a translation product of said gene, wherein said immunogenic or antigenic portion of a translation product is a polypeptide, and wherein said polypeptide elicits an antibody response in an animal, and wherein said polypeptide is immunospecifically bound by said antibody. Methods for generating antibodies are well known in the art. (see Harlow et al., Antibodies: A Laboratory Manual, Cold Spring Harbor Laboratory Press, Cold Spring Harbor, N.Y., 1988). The term “antibody”, as employed in the present invention, encompasses all forms of antibodies known in the art, such as polyclonal, monoclonal, chimeric, recombinatorial, anti-idiotypic, humanized, or single chain antibodies, as well as fragments thereof (see Dubel and Breitling, Recombinant Antibodies, Wiley-Liss, New York, N.Y., 1999). Antibodies of the present invention are useful, for instance, in a variety of diagnostic and therapeutic methods, based on state-in-the-art techniques (see Harlow and Lane, Using Antibodies: A Laboratory Manual, Cold Spring Harbor Laboratory Press, Cold Spring Harbor, N.Y., 1999 and Edwards R., Immunodiagnostics: A Practical Approach, Oxford University Press, Oxford, England, 1999) such as enzyme-immuno assays (e.g. enzyme-linked immunosorbent assay, ELISA), radioimmuno assays, chemoluminescence-immuno assays, Western-blot, immunoprecipitation and antibody microarrays. These methods involve the detection of translation products of the SCN2B gene, or fragments, or derivatives, or variants thereof.

In a preferred embodiment of the present invention, said antibodies can be used for detecting the pathological state of a cell in a sample from a subject, comprising immunocytochemical staining of said cell with said antibody, wherein an altered degree of staining, or an altered staining pattern in said cell compared to a cell representing a known health status indicates a pathological state of said cell. Preferably, the pathological state relates to a neurodegenerative disease, in particular to AD. Immunocytochemical staining of a cell can be carried out by a number of different experimental methods well known in the art. It might be preferred, however, to apply an automated method for the detection of antibody binding, wherein the determination of the degree of staining of a cell, or the determination of the cellular or subcellular staining pattern of a cell, or the topological distribution of an antigen on the cell surface or among organelles and other subcellular structures within the cell, are carried out according to the method described in U.S. Pat. No. 6,150,173.

Other features and advantages of the invention will be apparent from the following description of figures and examples which are illustrative only and not intended to limit the remainder of the disclosure in any way.

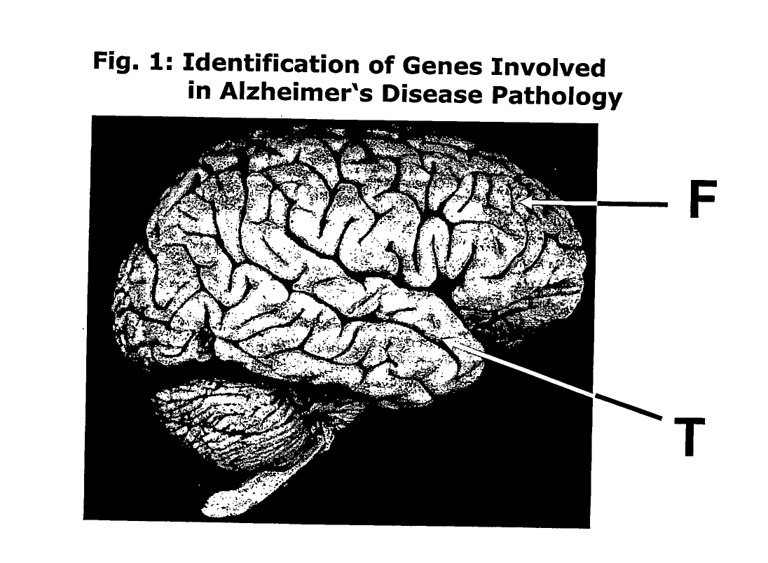

FIG. 1 depicts the brain regions with selective vulnerability to neuronal loss and degeneration in AD. Primarily, neurons within the inferior temporal lobe, the entorhinal cortex, the hippocampus, and the amygdala are subject to degenerative processes in AD (Terry et al., Annals of Neurology 1981, 10: 184-192). These brain regions are mostly involved in the processing of learning and memory functions. In contrast, neurons within the frontal cortex, the occipital cortex, and the cerebellum remain largely intact and preserved from neurodegenerative processes in AD. Brain tissues from the frontal cortex (F), the temporal cortex (T) and the hippocampus (H) of AD patients and healthy, age-matched control individuals were used for the herein disclosed examples. For illustrative purposes, the image of a normal healthy brain was taken from a publication by Strange (Brain Biochemistry and Brain Disorders, Oxford University Press, Oxford, 1992, p. 4).

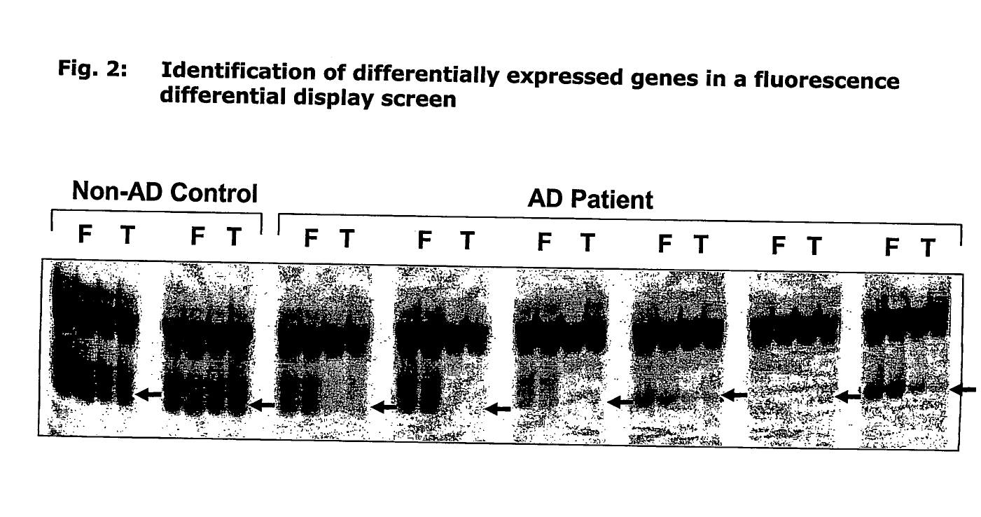

FIG. 2 discloses the initial identification of the differential expression of the gene coding for SCN2B in a fluorescence differential display screen. The figure shows a clipping of a large preparative fluorescent differential display gel. PCR products from the frontal cortex (F) and the temporal cortex (T) of two healthy control-subjects and six AD patients were loaded in duplicate onto a denaturing polyacrylamide gel (from left to right). PCR products were obtained by amplification of the individual cDNAs with the corresponding one-base-anchor oligonucleotide and the specific Cy3 labelled random primers. The arrow indicates the migration position where significant differences in intensity of the signals for a transcription product of the gene coding for SCN2B derived from frontal cortex as compared to the signals derived from the temporal cortex of AD patients exist. The differential expression reflects a down-regulation of SCN2B gene transcription in the temporal cortex compared to the frontal cortex of AD patients. Comparing the signals derived from temporal cortex and frontal cortex of healthy non-AD control subjects with each other, no difference in signal intensity, i.e. no altered expression level can be detected.

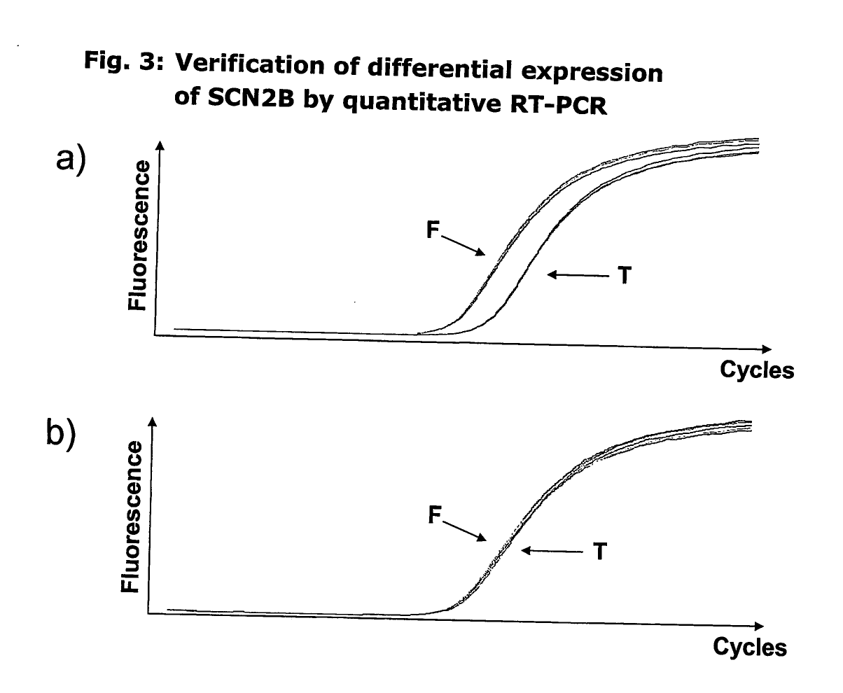

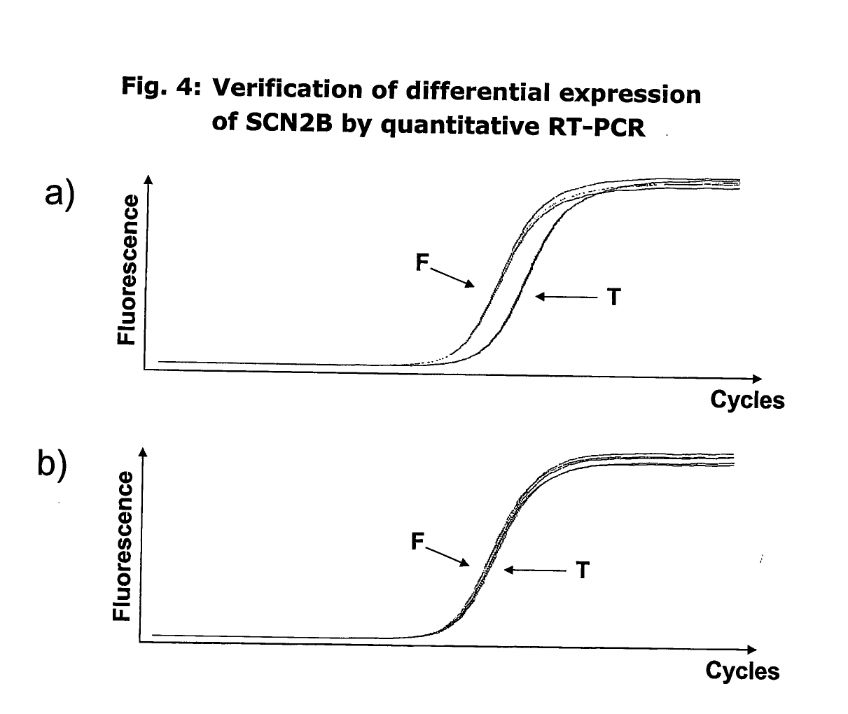

FIG. 3 and FIG. 4 illustrate the verification of the differential expression of the human SCN2B gene in AD brain tissues by quantitative RT-PCR analysis using two different set of primer pairs, primer set A (FIG. 3) and primer set B (FIG. 4). Quantification of RT-PCR products from RNA samples collected from the frontal cortex (F) and temporal cortex (T) of AD patients (FIG. 3a; 4a) and of a healthy, age-matched control individuals (FIG. 3b; 4b) was performed by the LightCycler rapid thermal cycling technique. The data were normalized to the combined average values of a set of standard genes which showed no significant differences in their gene expression levels. Said set of standard genes consisted of genes for the ribosomal protein S9, the transferrin receptor, GAPDH, cyclophilin B, and beta-actin. The figures depict the kinetics of amplification by plotting the cycle number against the amount of amplified material as measured by its fluorescence. Note that the amplification kinetics of the SCN2B cDNA from both the frontal and temporal cortices of a normal control individual during the exponential phase of the reaction are juxtaposed (FIG. 3b; 4b: arrows), whereas in AD (FIG. 3a; 4a: arrows), there is a significant separation of the corresponding curves, indicating a differential expression of the SCN2B gene in the two analyzed brain regions.

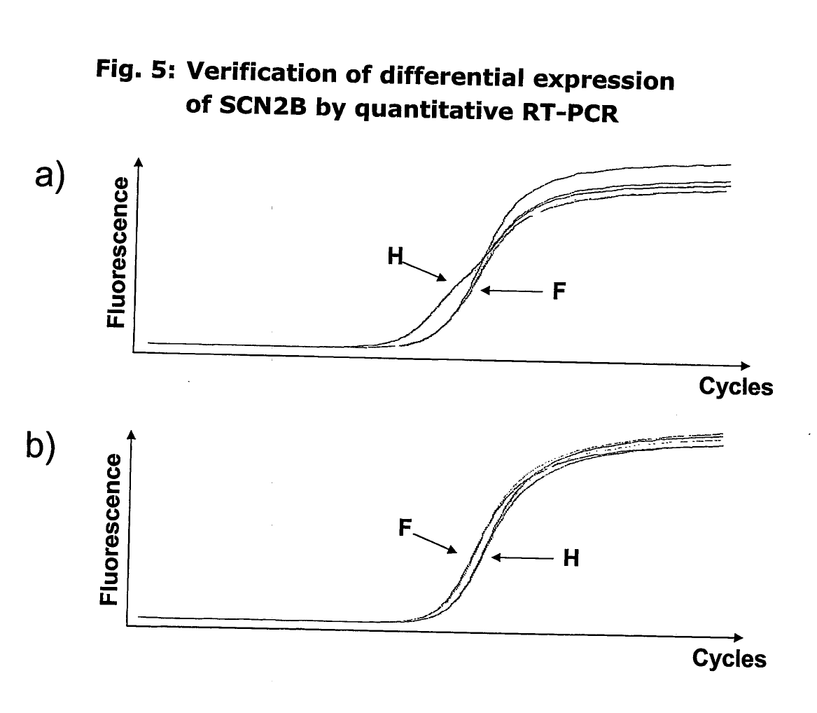

FIG. 5 illustrates the differential expression of the human SCN2B gene in AD brain tissues by quantitative RT-PCR analysis using primer set A. Quantification of RT-PCR products from RNA samples collected from the frontal cortex (F) and from the hippocampus (H) of AD patients (FIG. 5a) was performed by the LightCycler rapid thermal cycling technique. Likewise, samples of healthy, age-matched control individuals were compared (FIG. 5b). The data were normalized to the combined average values of a set of standard genes which showed no significant differences in their gene expression levels. Said set of standard genes consisted of genes for cyclophilin B, the ribosomal protein S9, the transferrin receptor, GAPDH, arid beta-actin. The figures depict the kinetics of amplification by plotting the cycle number against the amount of amplified material as measured by its fluorescence. Note that the amplification kinetics of SCN2B cDNAs from the frontal cortex and the hippocampus of a normal control individual during the exponential phase of the reaction are juxtaposed (FIG. 5b, arrowheads), whereas in Alzheimer's disease (FIG. 5a, arrowheads) there is a significant separation of the corresponding curves, indicating a differential expression of the gene coding for SCN2B in the respective analyzed brain regions, preferably a dysregulation of a transcription product of the human SCN2B gene, or a fragment, or derivative, or variant thereof, in the frontal cortex relative to the hippocampus.

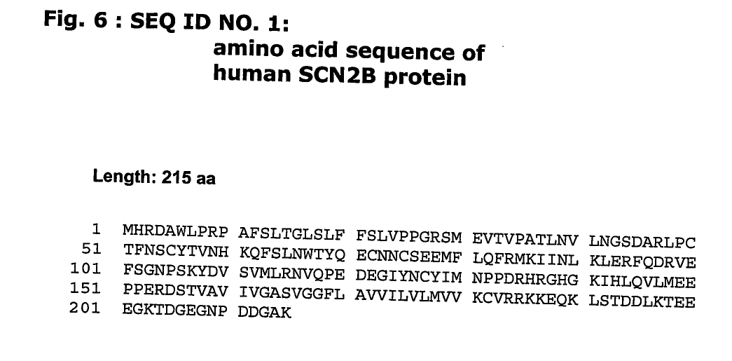

FIG. 6 discloses SEQ ID NO. 1; the amino acid sequence of the SCN2B protein. The full length human SCN2B protein comprises 215 amino acids.

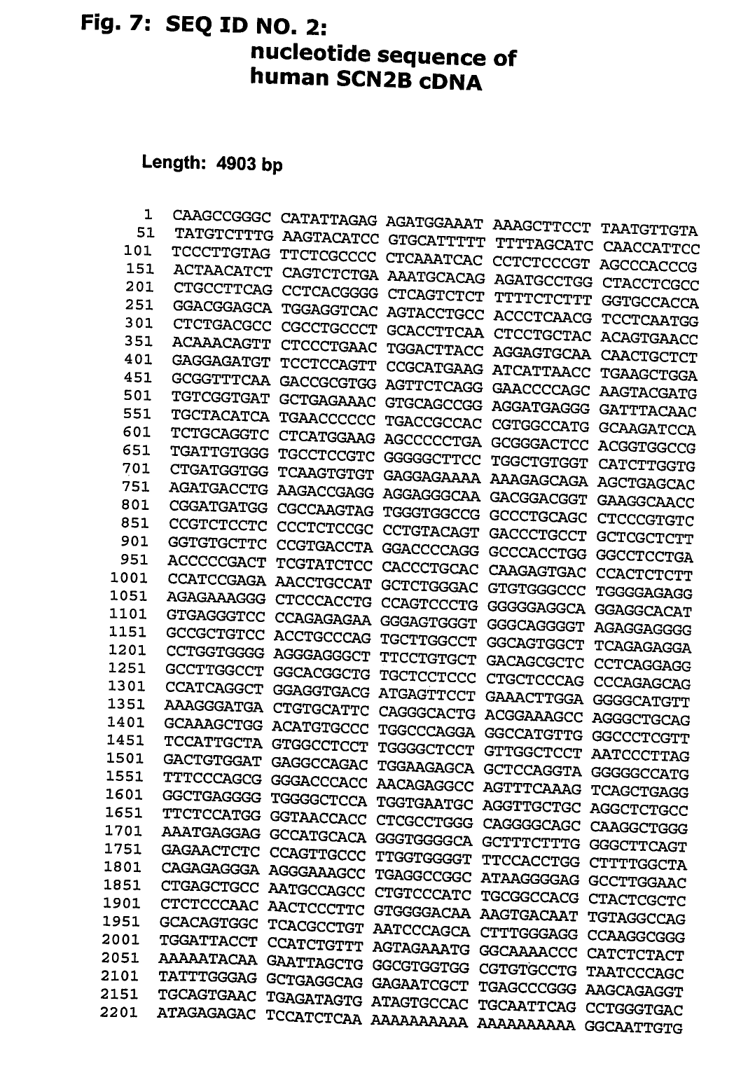

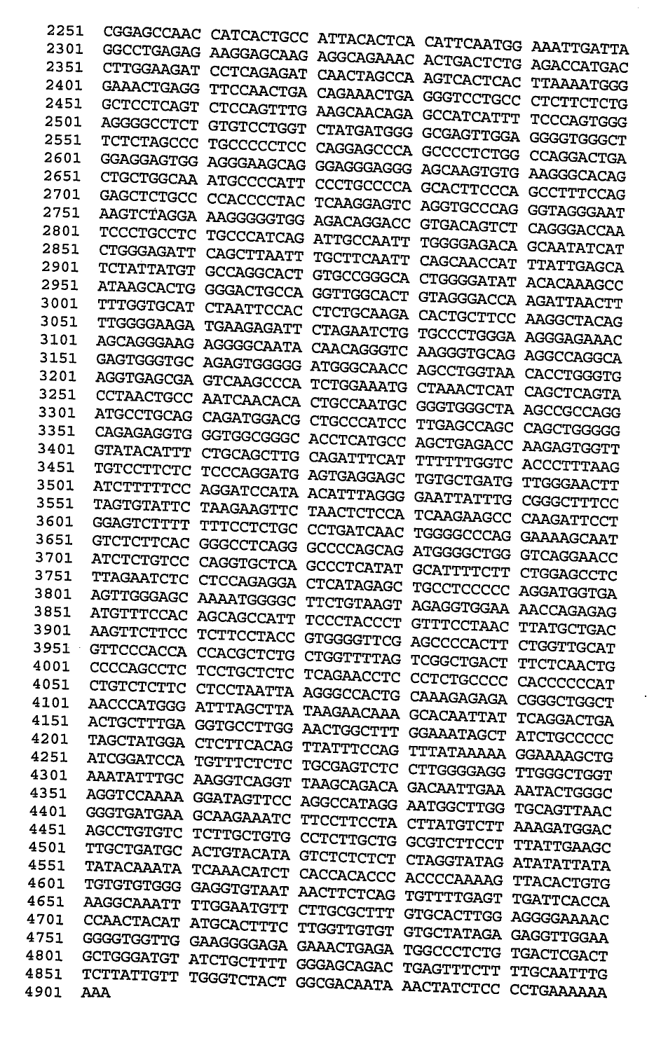

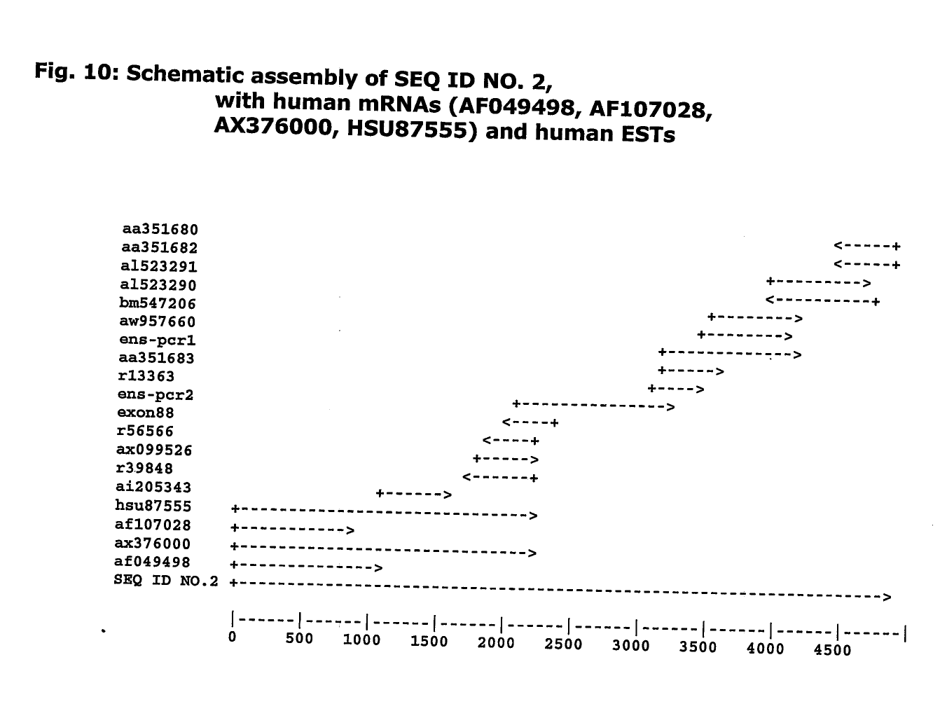

FIG. 7 shows SEQ ID NO. 2, the nucleotide sequence of the human SCN2B cDNA, comprising 4903 nucleotides, represented by a consensus sequence resulting from the assembly of a large redundant set of overlapping human nucleotide sequence fragments found in the Genbank human genomic database (see FIG. 10).

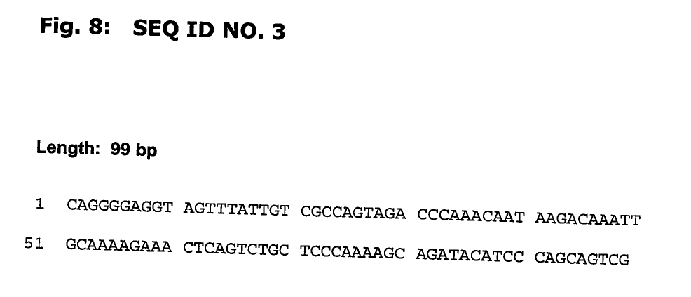

FIG. 8 depicts SEQ ID NO. 3, the nucleotide sequence of the 99 bp SCN2B cDNA fragment, identified and obtained by differential display (sequence in 5′ to 3′ direction).

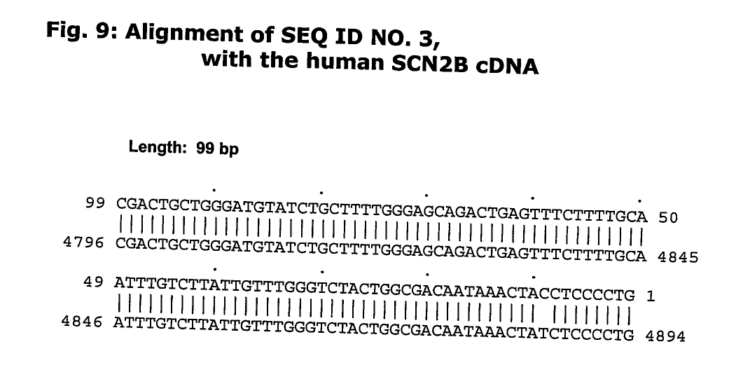

FIG. 9 outlines the sequence alignment of SEQ ID NO. 3 to the nucleotide sequence of the human SCN2B cDNA, SEQ ID NO. 2.

FIG. 10 schematically charts the assembly of SEQ ID NO. 2 from genomic database sequence fragments, constituting the SCN2B consensus cDNA sequence, a prolongated and corrected consensus sequence based on and derived from GenBank accession numbers AF049498, AF107028, AX376000, HSU87555 and the other indicated EST sequence fragments, and sequences derived from PCR amplification (ens-PCR1, ens-PCR2) with primers for the gene coding for SCN2B. Primer Set D (ens-PCR1, sequence nucleotides 3204 to 4182 of SEQ ID NO. 2): 5′-TGAGCGAGTCAAGCCCATCTGG-3′ and 5′-CCAAAGCCAGTTCCAAGGCACCTC-3′; Primer Set C (ens-PCR2, sequence nucleotides 2138 to 3230 of SEQ ID NO. 2): 5′-GGGAAGCAGAGGTTGCAGTGAAC-3′ and 5′-CATTTCCAGATGGGCTTGACTCGCTC-3′.

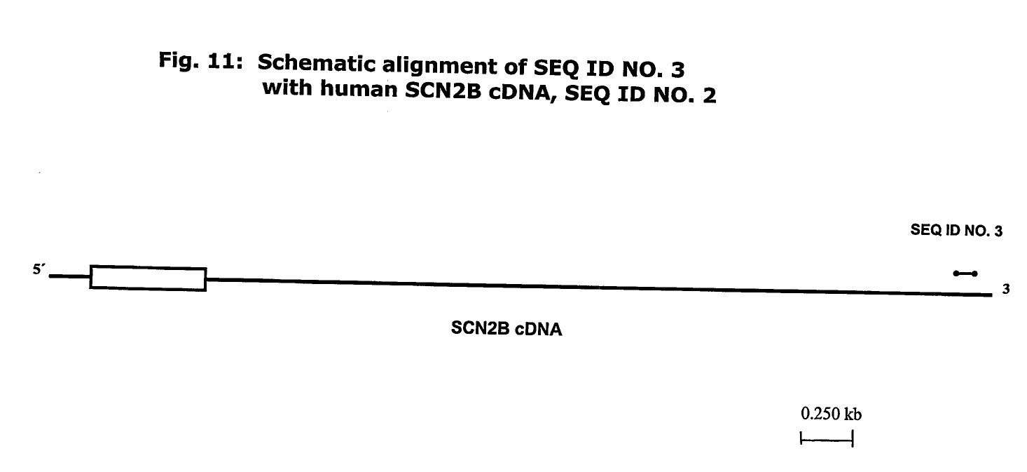

FIG. 11 shows a schematic alignment of SEQ ID NO. 3 with SCN2B cDNA. The open rectangle represents the SCN2B open reading frame, thin bars represent the 5′ and 3′ untranslated regions (UTRs).

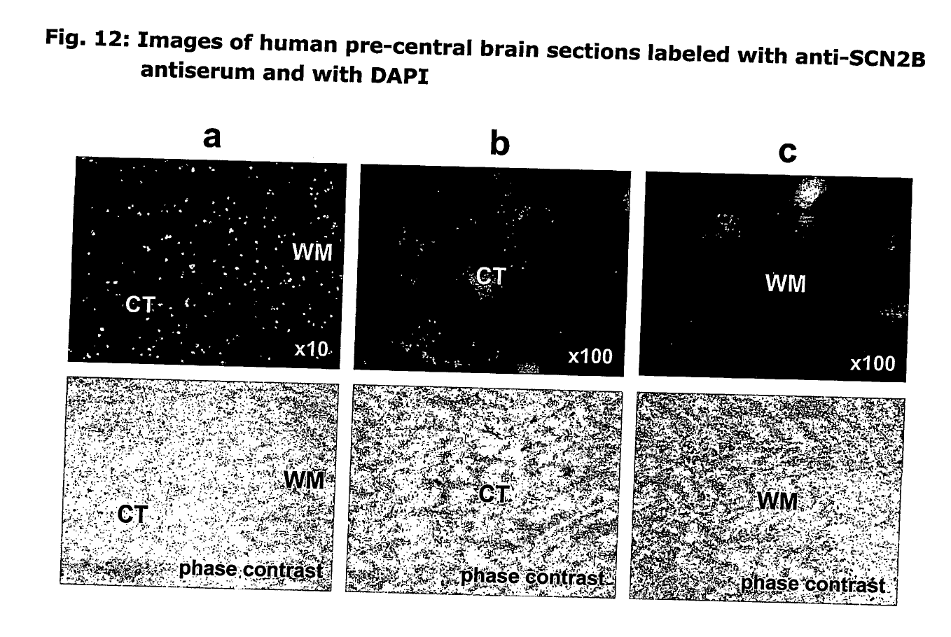

FIG. 12 depicts sections from human pre-central gyrus labeled with an affinity-purified rabbit polyclonal anti-SCN2B antiserum (green signals) raised against a peptide corresponding to amino acids 179 to 215 of SCN2B. Immunoreactivity of SCN2B was observed in both the cerebral cortex (CT) and the white matter (WM) (FIG. 12a, low magnification). A punctate pattern of SCN2B immunoreactivity decorating neuronal processes (dendritic spine-like) and a weaker and diffuse immunoreactivity of neuronal somata were observed (FIG. 12b, high magnification). FIG. 12c shows punctate immunoreactivity of glial cell bodies and nerve fibers. Blue signals indicate nuclei stained with DAPI.

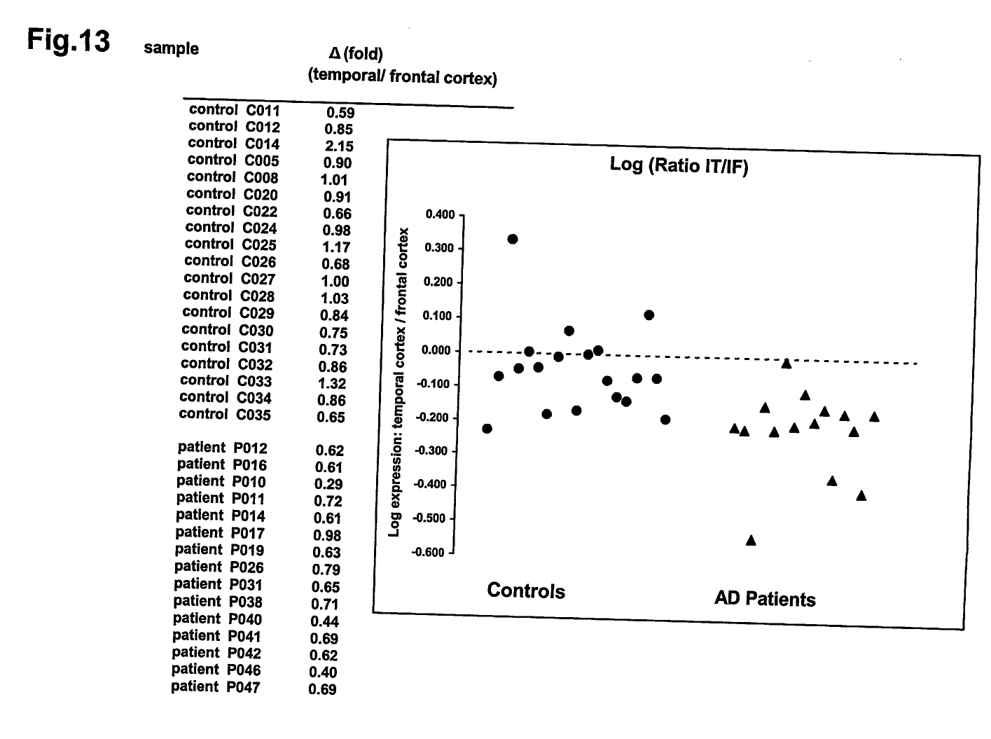

The table in FIG. 13 lists SCN2B gene expression levels in the temporal cortex relative to the frontal cortex in fifteen AD patients, herein identified by internal reference numbers P010, P011, P012, P014, P016, P017, P019, P026, P031, P038, P040, P041, P042, P046, P047 (0.29 to 0.98 fold) and nineteen healthy, age-matched control individuals, herein identified by internal reference numbers C005, C008, C011, C012, C014, C020, C022, C024, C025, C026, C027, C028, C029, C030, C031, C032, C033, C034, C035 (0.59 to 2.15 fold), Quantitative RT-PCR analysis was performed using primer pair set A. The values shown are calculated according to the formula described herein (see below). The scatter diagram visualizes individual logarithmic values of the temporal to frontal cortex regulation ratios, log(ratio IT/IF), in control samples (dots) and in AD patient samples (triangles), respectively.

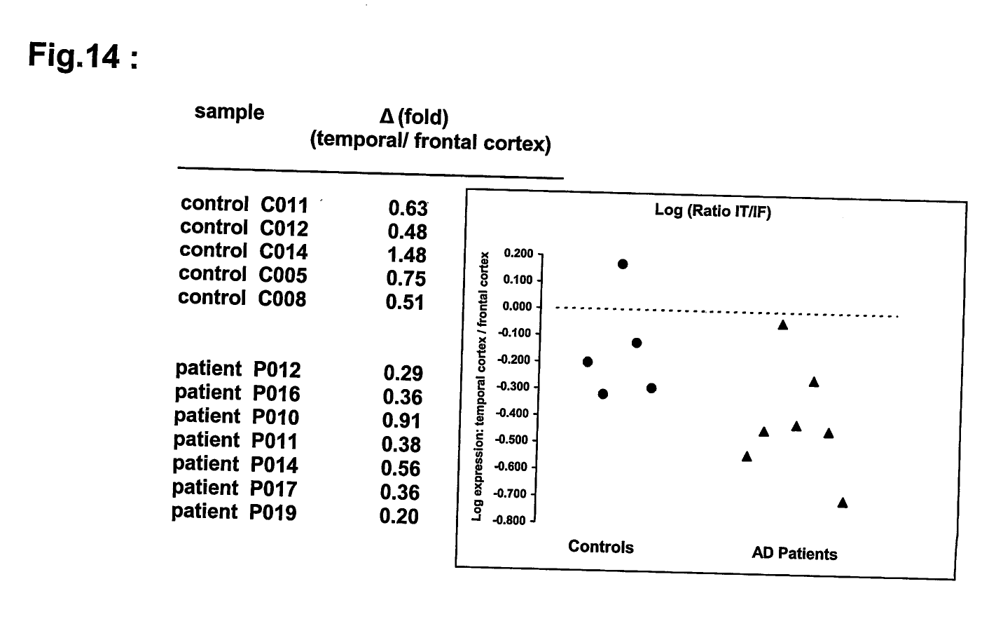

The table in FIG. 14 lists SCN2B gene expression levels in the temporal cortex relative to the frontal cortex in seven AD patients, herein identified by internal reference numbers P010, P011, P012, P014, P016, P017, P019 (0.20 to 0.91 fold) and five healthy, age-matched control individuals, herein identified by internal reference numbers C005, C008, C011, C012, C014 (0.48 to 1.48 fold). Quantitative RT-PCR analysis was performed using primer pair set B. The values shown are calculated according to the formula described herein (see below). The scatter diagram visualizes individual logarithmic values of the temporal to frontal cortex regulation ratios, log(ratio IT/IF), in control samples (dots) and in AD patient samples (triangles), respectively.

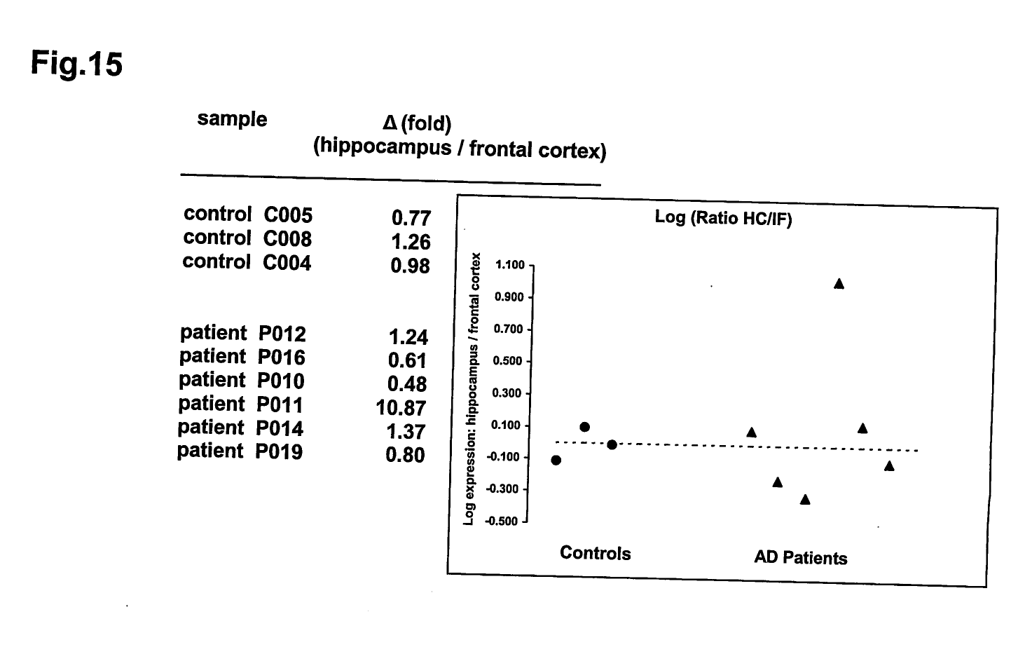

The table in FIG. 15 lists the gene expression levels in the hippocampus relative to the frontal cortex for the SCN2B gene in six Alzheimer's disease patients, herein identified by internal reference numbers P010, P011, P012, P014, P016, P019 (0.48 to 10.87 fold) and three healthy, age-matched control individuals, herein identified by internal reference numbers C004, C005, C008 (0.77 to 1.26 fold). Quantitative RT-PCR analysis was performed using primer pair set A. The values shown are calculated according to the formula described herein (see, below). The scatter diagram visualizes individual logarithmic values of the hippocampus to frontal cortex regulation ratios, log(ratio HC/IF), in control samples (dots) and in AD patient samples (triangles).

### EXAMPLE 1

(i) Brain Tissue Dissection from Patients with AD:

Brain tissues from AD patients and age-matched control subjects were collected within 6 hours post-mortem and immediately frozen on dry ice. Sample sections from each tissue were fixed in paraformaldehyde for histopathological confirmation of the diagnosis. Brain areas for differential expression analysis were identified (see FIG. 1.) and stored at −80° C. until RNA extractions were performed.

(ii) Isolation of Total mRNA:

Total RNA was extracted from post-mortem brain tissue by using the RNeasy kit (Qiagen) according to the manufacturer's protocol. The accurate RNA concentration and the RNA quality were determined with the DNA LabChip system using the Agilent 2100 Bioanalyzer (Agilent Technologies). For additional quality testing of the prepared RNA, i.e. exclusion of partial degradation and testing for DNA contamination, specifically designed intronic GAPDH oligonucleotides and genomic DNA as reference control were used to generate a melting curve with the LightCycler technology as described in the manufacturer's protocol (Roche).

(iii) cDNA Synthesis and Identification of Differentially Expressed Genes by Fluorescence Differential Display (FDD):

In order to identify changes in gene expression in different tissues we employed a modified and improved differential display (DD) screening method. The original DD screening method is known to those skilled in the art (Liang and Pardee, Science 1995, 267:1186-7). This technique compares two populations of RNA and provides clones of genes that are expressed in one population but not in the other. Several samples can be analyzed simultaneously and both up- and down-regulated genes can be identified in the same experiment. By adjusting and refining several steps in the DD method as well as modifying technical parameters, e.g. increasing redundancy, evaluating optimized reagents and conditions for reverse transcription of total RNA, optimizing polymerase chain reactions (PCR) and separation of the products thereof, a technique was developed which allows for highly reproducible and sensitive results. The applied and improved DD technique was described in detail by von der Kammer et al. (Nucleic Acids Research 1999, 27: 2211-2218). A set of 64 specifically designed random primers were developed (standard set) to achieve a statistically comprehensive analysis of all possible RNA species. Further, the method was modified to generate a preparative DD slab-gel technique, based on the use of fluorescently labelled primers. In the present invention, RNA populations from carefully selected post-mortem brain tissues (frontal and temporal cortex) of Alzheimer's disease patients and age-matched control subjects were compared. As starting material for the DD analysis we used total RNA, extracted as described above (ii). Equal amounts of 0.05 μg RNA each were transcribed into cDNA in 20 μl reactions containing 0.5 mM each dNTP, 1 μl Sensiscript™ Reverse Transcriptase and 1× RT buffer (Qiagen), 10 U RNase inhibitor (Qiagen) and 1 μM of either one-base-anchor oligonucleotides HT₁₁A, HT₁₁G or HT₁₁C (Liang et al., Nucleic Acids Research 1994, 22: 5763-5764; Zhao et al., Biotechniques 1995, 18:842-850). Reverse transcription was performed for 60 min at 37° C. with a final denaturation step at 93° C. for 5 min. 2 μl of the obtained cDNA each was subjected to a polymerase chain reaction (PCR) employing the corresponding one-base-anchor oligonucleotide (1 μM) along with either one of the Cy3 labelled random DD primers (1 μM), 1× GeneAmp PCR buffer (Applied Biosystems), 1.5 mM MgCl₂ (Applied Biosystems), 2 μM dNTP-Mix (dATP, dGTP, dCTP, dTTP Amersham Pharmacia Biotech), 5% DMSO (Sigma), 1 U AmpliTaq DNA Polymerase (Applied Biosystems) in a 20 μl final volume. PCR conditions were set as follows: one round at 94° C. for 30 sec for denaturing, cooling 1° C./sec down to 40° C., 40° C. for 4 min for low-stringency annealing of primer, heating 1° C./sec up to 72° C., 72° C. for 1 min for extension. This round was followed by 39 high-stringency-cycles: 94° C. for 30 sec, cooling 1° C./sec down to 60° C., 60° C. for 2 min, heating 1° C./sec up to 72° C., 72° C. for 1 min. One final step at 72° C. for 5 min was added to the last cycle (PCR cycler: Multi Cycler PTC 200, MJ Research). 8 μl DNA loading buffer were added to the 20 μl PCR product preparation, denatured for 5 min and kept on ice until loading onto a gel. 3.5 μl, each were separated on 0:4 mm thick, 6%-polyacrylamide (Long Ranger)/7 M urea sequencing gels in a slab-gel system (Hitachi Genetic Systems) at 2000 V, 60 W, 30 mA, for 1 h 40 min. Following completion of the electrophoresis, gels were scanned with a FMBIO II fluorescence-scanner (Hitachi Genetic Systems), using the appropriate FMBIO II Analysis 8.0 software. A full-scale picture was printed, differentially expressed bands marked, excised from the gel, transferred into 1.5 ml containers, overlayed with 200 μl, sterile water and kept at −20° C. until extraction.

Elution and reamplification of differential display products: The differential bands were extracted from the gel by boiling in 200 μl H₂O for 10 min, cooling down on ice and precipitation from the supernatant fluids by using ethanol (Merck) and glycogen/sodium acetate (Merck) at −20° C. over night, and subsequent centrifugation at 13.000 rpm for 25 min at 4° C. Pellets were washed twice in ice-cold ethanol (80%), resuspended in 10 mM Tris pH 8.3 (Merck) and dialysed against 10% glycerol (Merck) for 1 h at room temperature on a 0.025 μm VSWP membrane (Millipore). The obtained preparations were used as templates for reamplification by 15 high-stringency cycles in 25 μl PCR mixtures containing the corresponding primer pairs as used for the differential display PCR (see above) under identical conditions, with the exception of the initial round at 94° C. for 5 min, followed by 15 cycles of: 94° C. for 45 sec, 60° C. for 45 sec, ramp 1° C./sec to 70° C. for 45 sec, and one final step at 72° C. for 5 min.

Cloning and sequencing of differential display products: Re-amplified cDNAs-were analyzed with the DNA LabChip system (Agilent 2100 Bioanalyzer, Agilent Technologies) and were ligated into the pCR-Blunt II-TOPO vector and transformed into E.coli Top10 F′ cells (Zero Blunt TOPO PCR Cloning Kit, Invitrogen) according to the manufacturer's instructions. Cloned cDNA fragments were sequenced by commercially available sequencing facilities. The results of one such fluorescence differential display experiment for the human SCN2B gene are shown in FIG. 2.

(iv) Confirmation of Differential Expression by Quantitative RT-PCR:

Positive corroboration of differential expression of the gene coding for SCN2B was performed using the LightCycler technology (Roche). This technique features rapid thermal cyling for the polymerase chain reaction as well as real-time measurement of fluorescent signals during amplification and therefore allows for highly accurate quantification of RT-PCR products by using a kinetic, rather than an endpoint readout. The ratios of SCN2B cDNA from the temporal cortex and frontal cortex, and from the hippocampus and frontal cortex, respectively, were determined (relative quantification).

First, a standard curve was generated to determine the efficiency of the PCR with specific primers for the gene coding for SCN2B.

Primer Set A:
5′-GACTGCTGGGATGTATCTGCTTT-3′
and
5′-TTGTCGCCAGTAGACCCAAAC-3′.
Primer Set B:
5′-CCAAGGCTGGGAAATGAGG-3′
and
5′-CAAGGGCAACTGGGAGAGTTC-3′.

PCR amplification (95° C. and 1 sec, 56° C. and 5 sec, and 72° C. and 5 sec) was performed in a volume of 20 μl containing LightCycler-FastStart DNA Master SYBR Green I mix (contains FastStart Taq DNA polymerase, reaction buffer, dNTP mix with dUTP instead of dTTP, SYBR Green I dye, and 1 mM MgCl₂; Roche), 0.5 μM primers, 2 μl of a cDNA dilution series (final concentration of 40, 20, 10, 5, 1 and 0.5 ng human total brain cDNA; Clontech) and, depending on the primers used, additional 3 mM MgCl₂. Melting curve analysis revealed a single peak at approximately 86.5° C. with no visible primer dimers. Quality and size of the PCR product were determined with the DNA LabChip system (Agilent 2100 Bioanalyzer, Agilent Technologies). A single peak at the expected size of 82 bp for primer set A and 84 bp for primer set B was observed in the electropherogram of the sample. In an analogous manner, the PCR protocol was applied to determine the PCR efficiency of a set of reference genes which were selected as a reference standard for quantification In the present invention, the mean value of five such reference genes was determined: (1) cyclophilin B. using the specific primers 5′-ACTGAAGCACTACGGGCCTG-3′ and 5′-AGCCGTTGGTGTCTT TGCC-3′ except for MgCl₂ (an additional 1 mM was added instead of 3 mM). Melting curve analysis revealed a single peak at approximately 87° C. with no visible primer dimers. Agarose gel analysis of the PCR product showed one single band of the expected size (62 bp). (2) Ribosomal protein S9 (RPS9), using the specific primers 5′-GGTCAAATTTACCCTGGCCA-3′ and 5′-TCTCATCAAGCGTCAGCAGTTC-3′ (exception: additional 1 mM MgCl₂ was added instead of 3 mM). Melting curve analysis revealed a single peak at approximately 85° C. with no visible primer dimers. Agarose gel analysis of the PCR product showed one single band with the expected size (62 bp). (3) beta-actin, using the specific primers 5′-TGGAACGGTGAAGGTGACA-3′ and 5′-GGCMGCGGACTTCCTGTAA-3′. Melting curve analysis revealed a single peak at approximately 87° C. with no visible primer dimers. Agarose gel analysis of the PCR product showed one single band with the expected size (142 bp). (4) GAPDH, using the specific primers 5′-CGTCATGGGTGTGAMCCATG-3′ and 5′-GCTAAGCAGTTGOTGGTGCAG-3′. Melting curve analysis revealed a single peak at approximately 83° C. with no visible primer dimers. Agarose gel analysis of the PCR product showed one single band with the expected size (81 bp). (5) Transferrin receptor TRR, using the specific primers 5′-GTCGCTGGTCAGTTCGTGATT-3′ and 5′-AGCAGTTGGCTGTTGTACCTCTC-3′. Melting curve analysis revealed a single peak at approximately 83° C. with no visible primer dimers. Agarose gel analysis of the PCR product showed one single band with the expected size (80 bp).

For calculation of the values, first the logarithm of the cDNA concentration was plotted against the threshold cycle number Cₜ for the gene coding for SCN2B and the five reference standard genes. The slopes and the intercepts of the standard curves (i.e. linear regressions) were calculated for all genes. In a second step, cDNAs from frontal cortex and temporal cortex, and from frontal cortex and hippocampus, respectively, were analyzed in parallel and normalized to cyclophilin B. The Cₜ values were measured and converted to ng total brain cDNA using the corresponding standard curves:
10ˆ((Cₜvalue-intercept)/slope) [ng total brain cDNA]

The values for frontal and temporal cortex SCN2B cDNAs, and the values for frontal cortex and hippocampus SCN2B cDNAs, respectively, were normalized to cyclophilin B and the ratios were calculated according to formulas:

$$Ratio = \frac{SCN 2 B temporal [ ng ] / cyclophilin B temporal [ ng ]}{SCN 2 B frontal [ ng ] / cyclophilin B frontal [ ng ]}$$

$$Ratio = \frac{SCN 2 B hippocampus [ ng ] / cyclophilin B hippocampus [ ng ]}{SCN 2 B frontal [ ng ] / cyclophilin B frontal [ ng ]}$$

In a third step, the set of reference standard genes was analyzed in parallel to determine the mean average value of the temporal to frontal ratios, and of the hippocampal to frontal ratios, respectively, of expression levels of the reference standard genes for each individual brain sample. As cyclophilin B was analyzed in step 2 and step 3, and the ratio from one gene to another gene remained constant in different runs, it was possible to normalize the values for SCN2B to the mean average value of the set of reference standard genes instead of normalizing to one single gene alone. The calculation was performed by dividing the respective ratio shown above by the deviation of cyclophilin B from the mean value of all housekeeping genes. The results of such quantitative RT-PCR analysis for the SCN2B gene are shown in FIGS. 3, 4, and 5.

(v) Immunohistochemistry:

For immunofluorescence staining of SCN2B in human brain, frozen sections were prepared with a cryostat (Leica CM3050S) from post-mortem pre-central gyrus of a donor person and fixed in acetone for 10 min at room temperature. After washing in PBS, the sections were pre-incubated with blocking buffer (10% normal goat serum, 0.2% Triton X-100 in PBS) for 30 min and then incubated with affinity-purified rabbit anti-SCN2B antisera (1:20 to 1:40 diluted in blocking buffer; S-6811, Sigma) overnight at 4° C. After rinsing three times in 0.1% Triton X-100/PBS, the sections were incubated with FITC-conjugated goat anti-rabbit IgG antisera (1:150 diluted in 1% BSA/PBS) for 2 hours at room temperature and then again washed in PBS. Staining of the nuclei was performed by incubation of the sections with 5 μM DAPI in PBS for 3 min (blue signal). In order to block the autofluoresence of lipofuscin in human brain, the sections were treated with 1% Sudan Black B in 70% ethanol for 2-10 min at room temperature and then sequentially dipped in 70% ethanol, destined water and PBS. The sections were coverslipped with ‘Vectrashield’ mounting medium (Vector Laboratories, Burlingame, Calif.) and observed under an inverted microscope (1×81, Olympus Optical). The digital images were captured with the appropriate software (AnalySiS, Olympus Optical).

1. A method of diagnosing or prognosticating a neurodegenerative disease in a subject, or determining whether a subject is at increased risk of developing a neurodegenerative disease, comprising:
(a) determining a level or an activity of
(i) a transcription product of the gene coding for SCN2B, or
(ii) a translation product of the gene coding for SCN2B, or
(iii) a fragment, or derivative, or variant of said transcription or translation product,
in a sample from a subject; and
(b) comparing said level or said activity to a reference value representing a known disease or health status, thereby diagnosing or prognosticating a neurodegenerative disease in said subject, or determining whether said subject is at increased risk of developing said neurodegenerative disease.

2. The method according to claim 1 wherein said neurodegenerative disease is Alzheimer's disease.

3. A kit for diagnosing or prognosticating a neurodegenerative disease, in a subject, or determining the propensity or predisposition of a subject to develop such a disease, said kit comprising:
(a) at least one reagent which is selected from the group consisting of (i) reagents that selectively detect a transcription product of the gene coding for SCN2B; and (ii) reagents that selectively detect a translation product of the gene coding for SCN2B; and
(b) an instruction for diagnosing or prognosticating a neurodegenerative disease, or determining the propensity or predisposition of a subject to develop such a disease by (i) detecting a level, or an activity, or both said level and said activity, of said transcription product and/or said translation product of the gene coding for SCN2B, in a sample from said subject; and (ii) diagnosing or prognosticating a neurodegenerative disease, or determining the propensity or predisposition of said subject to develop such a disease, wherein a varied level, or activity, or both said level and said activity, of said transcription product or said translation product compared to a reference value representing a known health status; or a level, or activity, or both said level and said activity, of said transcription product or said translation product similar or equal to a reference value representing a known disease status indicates a diagnosis or prognosis of a neurodegenerative disease, or an increased propensity or predisposition of developing such a disease.

4. A compound which is a modulator of an activity or of a level of at least one substance which is selected from the group consisting of
(i) the gene coding for SCN2B,
(ii) a transcription product of the gene coding for SCN2B,
(iii) a translation product of the gene coding for SCN2B, and
(iv) a fragment, or derivative, or variant of (i) to (iii).

5. A recombinant, non-human animal comprising a non-native gene sequence coding for SCN2B or a fragment, or a derivative, or a variant thereof, said non-human animal being obtainable by:
(i) providing a gene targeting construct comprising a non-native gene sequence and a selectable marker sequence,
(ii) introducing said targeting construct into a stem cell of a non-human animal,
(iii) introducing said stem cell of a non-human animal into a non-human embryo,
(iv) transplanting said embryo into a pseudopregnant non-human animal,
(v) allowing said embryo to develop to term,
(vi) identifying a genetically altered non-human animal whose genome comprises a modification of said gene sequence in both alleles, and
(vii) breeding the genetically altered non-human animal of step (vi) to obtain a genetically altered non-human animal whose genome comprises a modification of said endogenous gene, wherein said modification results in said non-human animal exhibiting a predisposition to developing symptoms of a neurodegenerative disease or related diseases or disorders.

6. A method for screening for a modulator of of one or more substances selected from the group consisting of
(i) the gene coding for SCN2B,
(ii) a transcription product of the gene coding for SCN2B,
(iii) a translation product of the gene coding for SCN2B, and
(iv) a fragment, or derivative, or variant of (i) to (iii), said method comprising:
(a) contacting a cell with a test compound;
(b) measuring the activity or level of one or more substances recited in (i) to (iv);
(c) measuring the activity or level of one or more substances recited in (i) to (iv) in a control cell not contacted with said test compound; and
(d) comparing the levels or activities of the substance in the cells of step (b) and (c), wherein an alteration in the activity and/or level of substances in the contacted cells indicates that the test compound is a modulator of neurodegenerative diseases or disorders.

7. A method of screening for a modulator of neurodegenerative diseases, of one or more substances selected from the group consisting of
(i) the gene coding for SCN2B,
(ii) a transcription product of the gene coding for SCN2B,
(iii) a translation product of the gene coding for SCN2B, and
(v) a fragment, or derivative, or variant of (i) to (iii),
said method comprising:
(a) administering a test compound to a non-human test animal which is predisposed to developing or has already developed symptoms of a neurodegenerative disease or related diseases or disorders in respect of the substances recited in (i) to (iv);
(b) measuring the activity or level of one or more substances recited in (i) to (iv);
(c) measuring the activity or level of one or more substances recited in (i) or (iv) in a matched non-human control animal which is predisposed to developing or has already developed symptoms of a neurodegenerative disease or related diseases or disorders in respect to the substances recited in (i) to (iv) and to which non-human animal no such test compound has been administered;
(d) comparing the activity or level of the substance in the non-human animals of step (b) and (c), wherein an alteration in the activity or level of substances in the non-human test animal indicates that the test compound is a modulator of said neurodegenerative diseases or disorders.

8. The method according to claim 7 wherein said non-human test animal or said non-human control animal is a recombinant non-human animal which expresses SCN2B, or a fragment, or a derivative, or a variant thereof, under the control of a transcriptional control element which is not the native SCN2B gene transcriptional control element.

9. An method for assaying an SCN2B translation product, or a fragment, or derivative, or variant thereof, said method comprising the steps of:
(i) adding a liquid suspension of an SCN2B translation product, or a fragment, or derivative, or variant thereof, to a plurality of containers;
(ii) adding a one or more detectable compounds to be screened for said binding to said plurality of containers;
(iii) incubating said SCN2B translation product, or said fragment, or derivative, or variant thereof, and said detectable compounds;
(iv) measuring amounts of said one or more detectable compounds associated with said SCN2B translation product, or with said fragment, or derivative, or variant thereof; and
(v) determining the degree of binding by one or more of said compounds to said SCN2B translation product, or said fragment, or derivative, or variant thereof.

10. An isolated protein molecule, a translation product of the gene coding for SCN2B, SEQ ID NO. 1, or a fragment, or derivative, or variant thereof, for use as a diagnostic target for detecting a neurodegenerative disease, preferably Alzheimer's disease.

11. An isolated protein molecule, which is a translation product of the gene coding for SCN2B, SEQ ID NO. 1, or a fragment, or derivative, or variant thereof, for use as a screening target for reagents or compounds preventing, or treating, or ameliorating a neurodegenerative disease, preferably Alzheimer's disease.

12. A method for detecting the pathological state of a cell from a subject comprising:
immunocytochemically staining of a cell with an antibody, wherein said antibody is specifically immunoreactive with a translation product of a gene coding for SCN2B, SEQ ID NO. 1, or a fragment, or derivative, or variant thereof, and wherein an altered degree of staining, or an altered staining pattern in said cell compared to a cell representing a known health status, indicates a pathological state of said cell.

13. The kit according to claim 3, wherein said neurodegenerative disease is Alzheimer's disease.

14. The method of claim 6, wherein said neurodegenerative disease is Alzheimer's disease.

15. The method of claim 7, wherein said neurodegenerative disease is Alzheimer's disease.

16. The method of claim 9, wherein said one or more detectable compounds are fluorescently labeled compounds.


SEQUENCE LISTING (24 sequences)

SEQ ID NO: 1 - protein, 215 aa, Homo sapiens
MHRDAWLPRPAFSLTGLSLFFSLVPPGRSMEVTVPATLNVLNGSDARLPCTFNSCYTVNHKQFSLNWTYQECNNCSEEMFLQFRMKIINLKLERFQDRVEFSGNPSKYDVSVMLRNVQPEDEGIYNCYIMNPPDRHRGHGKIHLQVLMEEPPERDSTVAVIVGASVGGFLAVVILVLMVVKCVRRKKEQKLSTDDLKTEEEGKTDGEGNPDDGAK

SEQ ID NO: 2 - DNA, 4903 bp, Artificial Sequence
  cDNA of the Human SCN2B Gene
caagccgggccatattagagagatggaaataaagcttccttaatgttgtatatgtctttgaagtacatccgtgcatttttttttagcatccaaccattcctcccttgtagttctcgccccctcaaatcaccctctcccgtagcccacccgactaacatctcagtctctgaaaatgcacagagatgcctggctacctcgccctgccttcagcctcacggggctcagtctctttttctctttggtgccaccaggacggagcatggaggtcacagtacctgccaccctcaacgtcctcaatggctctgacgcccgcctgccctgcaccttcaactcctgctacacagtgaaccacaaacagttctccctgaactggacttaccaggagtgcaacaactgctctgaggagatgttcctccagttccgcatgaagatcattaacctgaagctggagcggtttcaagaccgcgtggagttctcagggaaccccagcaagtacgatgtgtcggtgatgctgagaaacgtgcagccggaggatgaggggatttacaactgctacatcatgaacccccctgaccgccaccgtggccatggcaagatccatctgcaggtcctcatggaagagccccctgagcgggactccacggtggccgtgattgtgggtgcctccgtcgggggcttcctggctgtggtcatcttggtgctgatggtggtcaagtgtgtgaggagaaaaaaagagcagaagctgagcacagatgacctgaagaccgaggaggagggcaagacggacggtgaaggcaacccggatgatggcgccaagtagtgggtggccggccctgcagcctcccgtgtcccgtctcctcccctctccgccctgtacagtgaccctgcctgctcgctcttggtgtgcttcccgtgacctaggaccccagggcccacctggggcctcctgaacccccgacttcgtatctcccaccctgcaccaagagtgacccactctcttccatccgagaaacctgccatgctctgggacgtgtgggccctggggagaggagagaaagggctcccacctgccagtccctggggggaggcaggaggcacatgtgagggtccccagagagaagggagtgggtgggcaggggtagaggagggggccgctgtccacctgcccagtgcttggcctggcagtggcttcagagaggacctggtggggagggagggctttcctgtgctgacagcgctccctcaggagggccttggcctggcacggctgtgctcctcccctgctcccagcccagagcagccatcaggctggaggtgacgatgagttcctgaaacttggaggggcatgttaaagggatgactgtgcattccagggcactgacggaaagccagggctgcaggcaaagctggacatgtgccctggcccaggaggccatgttgggccctcgtttccattgctagtggcctccttggggctcctgttggctcctaatcccttaggactgtggatgaggccagactggaagagcagctccaggtagggggccatgtttcccagcggggacccaccaacagaggccagtttcaaagtcagctgaggggctgaggggtggggctccatggtgaatgcaggttgctgcaggctctgccttctccatggggtaaccaccctcgcctgggcaggggcagccaaggctgggaaatgaggaggccatgcacagggtggggcagctttctttggggcttcagtgagaactctcccagttgcccttggtggggtttccacctggcttttggctacagagagggaagggaaagcctgaggccggcataaggggaggccttggaacctgagctgccaatgccagccctgtcccatctgcggccacgctactcgctcctctcccaacaactcccttcgtggggacaaaagtgacaattgtaggccaggcacagtggctcacgcctgtaatcccagcactttgggaggccaaggcgggtggattacctccatctgtttagtagaaatgggcaaaaccccatctctactaaaaatacaagaattagctgggcgtggtggcgtgtgcctgtaatcccagctatttgggaggctgaggcaggagaatcgcttgagcccgggaagcagaggttgcagtgaactgagatagtgatagtgccactgcaattcagcctgggtgacatagagagactccatctcaaaaaaaaaaaaaaaaaaaaaaggcaattgtgcggagccaaccatcactgccattacactcacattcaatggaaattgattaggcctgagagaaggagcaagaggcagaaacactgactctgagaccatgaccttggaagatcctcagagatcaactagccaagtcactcacttaaaatggggaaactgaggttccaactgacagaaactgagggtcctgccctcttctctggctcctcagtctccagtttgaagcaacagagccatcattttcccagtgggaggggcctctgtgtcctggtctatgatggggcgagttggaggggtgggcttctctagccctgccccctcccaggagcccagcccctctggccaggactgaggaggagtggagggaagcagggagggagggagcaagtgtgaagggcacagctgctggcaaatgccccattccctgccccagcacttcccagcctttccaggagctctgccccacccctactcaaggagtcaggtgcccagggtagggaataagtctaggaaagggggtggagacaggaccgtgacagtctcagggaccaatccctgcctctgcccatcagattgccaatttggggagacagcaatatcatctgggagattcagcttaatttgcttcaattcagcaaccatttattgagcatctattatgtgccaggcactgtgccgggcactggggatatacacaaagccataagcactggggactgccaggttggcactgtagggaccaagattaactttttggtgcatctaattccacctctgcaagacactgcttccaaggctacagttggggaagatgaagagattctagaatctgtgccctgggaagggagaaacagcagggaagaggggcaatacaacagggtcaagggtgcagaggccaggcagagtgggtgcagagtgggggatgggcaaccagcctggtaacacctgggtgaggtgagcgagtcaagcccatctggaaatgctaaactcatcagctcagtacctaactgccaatcaacacactgccaatgcgggtgggctaagccgccaggatgcctgcagcagatggacgctgcccatccttgagccagccagctgggggcagagaggtgggtggcgggcacctcatgccagctgagaccaagagtggttgtatacatttctgcagcttgcagatttcatttttttggtcaccctttaagtgtccttctctcccaggatgagtgaggagctgtgctgatgttgggaacttatctttttccaggatccataacatttaggggaattatttgcgggctttcctagtgtattctaagaagttctaactctccatcaagaagcccaagattcctggagtctttttttcctctgccctgatcaactggggcccaggaaaagcaatgtctcttcacgggcctcagggccccagcagatggggctgggtcaggaaccatctctgtcccaggtgctcagccctcatatgcattttcttctggagcctcttagaatctcctccagaggactcatagagctgcctcccccaggatggtgaagttgggagcaaaatggggcttctgtaagtagaggtggaaaaccagagagatgtttccacagcagccatttccctaccctgtttcctaacttatgctgacaagttcttcctcttcctaccgtggggttcgagccccacttctggttgcatgttcccaccaccacgctctgctggttttagtcggctgactttctcaactgccccagcctctcctgctctctcagaacctccctctgcccccaccccccatctgtctcttcctcctaattaagggccactgcaaagagagacgggctggctaacccatgggatttagcttataagaacaaagcacaattattcaggactgaactgctttgaggtgccttggaactggctttggaaatagctatctgccccctagctatggactcttcacagttatttccagtttataaaaaggaaaagctgatcggatccatgtttctctctgcgagtctccttggggaggttgggctggtaaatatttgcaaggtcaggttaagcagacagacaattgaaaatactgggcaggtccaaaaggatagttccaggccataggaatggcttggtgcagttaacgggtgatgaagcaagaaatcttccttcctacttatgtcttaaagatggacagcctgtgtctcttgctgtgcctcttgctggcgtcttcctttattgaagcttgctgatgcactgtacatagtctctctctctaggtatagatatattatatatacaaatatcaaacatctcaccacacccaccccaaaagttacactgtgtgtgtgtggggaggtgtaataacttctcagtgttttgagttgattcaccaaaggcaaattttggaatgttcttgcgctttgtgcacttggaggggaaaacccaactacatatgcactttcttggttgtgtgtgctatagagaggttggaaggggtggttggaaggggagagaaactgagatggccctctgtgactcgactgctgggatgtatctgcttttgggagcagactgagtttcttttgcaatttgtcttattgtttgggtctactggcgacaataaactatctcccctgaaaaaaaaa

SEQ ID NO: 3 - DNA, 99 bp, Artificial Sequence
  cDNA Fragment of the Human SCN2B Gene
caggggaggtagtttattgtcgccagtagacccaaacaataagacaaattgcaaaagaaactcagtctgctcccaaaagcagatacatcccagcagtcg

SEQ ID NO: 4 - DNA, 22 bp, Artificial Sequence
  Primer of the Primer Set D for the SCN2B Gene
tgagcgagtcaagcccatctgg

SEQ ID NO: 5 - DNA, 23 bp, Artificial Sequence
  Primer of the Primer Set D for the SCN2B Gene
caaagccagttccaaggcacctc

SEQ ID NO: 6 - DNA, 23 bp, Artificial Sequence
  Primer of the Primer Set C for the SCN2B Gene
gggaagcagaggttgcagtgaac

SEQ ID NO: 7 - DNA, 26 bp, Artificial Sequence
  Primer of the Primer Set C for the SCN2B Gene
catttccagatgggcttgactcgctc

SEQ ID NO: 8 - DNA, 13 bp, Artificial Sequence
  One-base-anchor Oligonucleotide
httttttttttta

SEQ ID NO: 9 - DNA, 13 bp, Artificial Sequence
  One-base-anchor Oligonucleotide
htttttttttttg

SEQ ID NO: 10 - DNA, 13 bp, Artificial Sequence
  One-base-anchor Oligonucleotide
htttttttttttc

SEQ ID NO: 11 - DNA, 23 bp, Artificial Sequence
  Primer of the Primer Set A for the SCN2B Gene
gactgctgggatgtatctgcttt

SEQ ID NO: 12 - DNA, 21 bp, Artificial Sequence
  Primer of the Primer Set A for the SCN2B Gene
ttgtcgccagtagacccaaac

SEQ ID NO: 13 - DNA, 19 bp, Artificial Sequence
  Primer of the Primer Set B for the SCN2B Gene
ccaaggctgggaaatgagg

SEQ ID NO: 14 - DNA, 21 bp, Artificial Sequence
  Primer of the Primer Set B for the SCN2B Gene
caagggcaactgggagagttc

SEQ ID NO: 15 - DNA, 20 bp, Artificial Sequence
  Primer for the Cyclophilin B Gene
actgaagcactacgggcctg

SEQ ID NO: 16 - DNA, 19 bp, Artificial Sequence
  Primer for the Cyclophilin B Gene
agccgttggtgtctttgcc

SEQ ID NO: 17 - DNA, 20 bp, Artificial Sequence
  Primer for the Gene of the Ribosomal Protein S9
ggtcaaatttaccctggcca

SEQ ID NO: 18 - DNA, 22 bp, Artificial Sequence
  Primer for the Gene of the Ribosomal Protein S9
tctcatcaagcgtcagcagttc

SEQ ID NO: 19 - DNA, 19 bp, Artificial Sequence
  Primer for the Beta-Actin Gene
tggaacggtgaaggtgaca

SEQ ID NO: 20 - DNA, 19 bp, Artificial Sequence
  Primer for the Beta-Actin Gene
ggcaagggacttcctgtaa

SEQ ID NO: 21 - DNA, 20 bp, Artificial Sequence
  Primer for the GAPDH Gene
cgtcatgggtgtgaaccatg

SEQ ID NO: 22 - DNA, 21 bp, Artificial Sequence
  Primer for the GAPDH Gene
gctaagcagttggtggtgcag

SEQ ID NO: 23 - DNA, 21 bp, Artificial Sequence
  Primer for the Gene of the Transferrin Receptor
gtcgctggtcagttcgtgatt

SEQ ID NO: 24 - DNA, 23 bp, Artificial Sequence
  Primer for the Gene of the Transferrin Receptor
agcagttggctgttgtacctctc